(19) United States
(12) Patent Application Publication
(10) Pub. No.: US 2009/0123392 A1
(43) Pub. Date: May 14, 2009

(54) FRAGRANCE ACCORDS TO COMBAT THE PERCEPTION OF BODY ODOR

(75) Inventors: Dirk Braun, Cedar Grove, NJ (US); Keith McDermott, Bound Brook, NJ (US); James G. Clothier, Ridgewood, NJ (US)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/267,942

(22) Filed: Nov. 10, 2008

Related U.S. Application Data
(60) Provisional application No. 60/987,187, filed on Nov. 12, 2007.

(51) Int. Cl. A61K 9/12; A61K 8/37; A61Q 15/00

(52) U.S. Cl. 424/47; 424/65

(57) ABSTRACT

The present invention concerns an accord, comprising, essentially consisting or consisting of:
i) ethylene brassylate,
(ii) alpha-isomethyl ionone,
iii) 2-methyl-5-phenylpentan-1-ol, and
iv) menthyl acetate,
   as well as possibly one, two, three, four or all of components v) to ix):
v) alpha-n-methyl ionone,
vi) isomenthyl acetate,
vii) vanillin,
viii) neomenthyl acetate,
ix) neoisomenthyl acetate.

### CROSS-REFERENCE TO RELATED APPLICATION

This application claims benefit of priority to U.S. Provisional Application 60/987,187, filed on Nov. 12, 2007, which is incorporated herein by reference in its entirety.

The invention concerns certain fragrance mixtures (accords) and their application in generating a sense of hygiene and/or cleanliness. In this connection the invention concerns in particular corresponding applications, in which the sense of hygiene and/or cleanliness is generated by the interaction of the accord with a body odor, wherein the body odor is weakened and/or masked. Here the sense of hygiene and/or cleanliness can be perceived both by the user him/herself and the other people around him/her.

Furthermore, the invention concerns perfume oils and cosmetic preparations, comprising corresponding accords and the use of these fragrance mixtures to generate a sense of hygiene and/or cleanliness. The invention also concerns corresponding methods.

The sense of hygiene and cleanliness is here generally triggered by stimuli in the nasal cavity. Such a sense also occurs in particular if pleasant smells are perceived generally or reinforced, or unpleasant smells, in particular those associated with lack of (personal) hygiene, such as the smell of sweat, are not or are no longer perceived.

Deodorants and antiperspirants (antiaspirants) are cosmetic preparations which are used for prevention and removal of unpleasant body odor, in particular the smell of underarm sweat. They provide the user with the sense of and radiate hygiene and cleanliness.

The way in which deodorants and antiperspirants work can be based on a variety of mechanisms. Thus various active substances are used, such as
substances with a deodorizing effect (deodorants):
   antimicrobial acting substances, enzyme inhibiting substances, odor absorbing substances, odor neutralizing substances and odor masking substances; and
sweat-inhibiting substances (antiperspirants, antitranspirants).

The effect of these substances is predominantly intended, over as long a period as possible, to prevent the development of unpleasant body odor or the perception of this.

In the case of an existing smell of sweat it is desirable if in particular the deodorants used by the user result in the immediate start to, and the fastest possible perception of, the sense of hygiene and cleanliness. Such a rapid effect can only be achieved with odor masking substances, i.e. by perfumes with a correspondingly strong odorous effect or by the olfactory perception of masking substances. Because of their strength or limited (specific) method of working these substances can, however, often only be used in quite specific odor types, which are not always associated by all consumers with a sense of hygiene and cleanliness.

Numerous proposals for combating body odor and other unpleasant smells can be found in the literature.

EP O 780 132 describes the combating of unpleasant smells, especially cooking and waste smells, tobacco odor and the smell of excrement, with particular fragrance mixtures. The mixtures described therein comprise approximately 20-60% by weight of musk fragrances, approximately 30-70% by weight citrus fragrances and between 1 and 20% by weight peppermint odor substances. Galaxolide®, Tonalid®, Pentadecanolid und Exaltolid® are mentioned as preferred musk fragrances.

In WO 98/27261, JP-A 10 120 541, U.S. Pat. No. 5,559,271, JP-A 05 058 869, JP-A 2 726 846, JP-A 03 029 280 and JP-A 03 044 781 menthyl acetate, or 1-menthyl acetate is described as an ingredient of perfume mixtures suitable for combating unpleasant odors.

WO 01/43784 discloses the use of certain esters, in particular isomenthyl esters such as isomenthyl acetate, for masking unpleasant odors of various kinds.

EP 1 113 05 describes a mixture to counter the smell of human sweat as an additive to a perfume, and mentions the following components, inter alia: beta-naphthylmethylether, methyl-beta-naphthylketone, benzylacetone, gamma-methyl ionone, geranonitrile, ethylene brassylate, cyclohexadecen-5-one-1 and/or 1-cycloheptadecen-10-one.

EP 0 771 561 describes Rosaphen™ as an antimycotic active substance and discloses deo formulations containing Rosaphen™.

EP 1 157 687 describes antimicrobial deo formulations containing Rosaphen™ as the active substances which are in particular active against body odor.

JP 06-179610 describes fragrance mixtures effective against human sweat which from a group comprising at least one alcohol such as citronellol, geraniol, tetrahydrogeraniol, farnesol and/or phenyl ethyl dimethyl carbinol, contain at least one ester such as ethylene brassylate, coumarin, gamma-decalactone, citronellyl acetate, cis-3-hexenylsalicylate, and possibly further compounds from the class of ketones or aldehydes or their acetates or ketals from the group cis-jasmone, heliotropin, moschus ketone, methylnonylacetaldehyde. Apart from this the fragrance mixtures effective against sweat must contain further components which are selected from a number of etheric oils.

JP 2003081804 describes a formulation which is suitable as a bath essence and masks body odor. For the fragrancing, by way of example, lemons, alpha-pines, cis-3-hexen-1-ol, 1-penten-3-ol, myrcenol, octanal, nonanal, sinensal, amyl acetate, 3-phenylpropyl alcohol, citronellol, jasmal, allylcyclohexyl-oxyacetate, geranyl acetate, iso-butylpropionate, 10-undecenal, dodecanal, tripral, L-carvone, menthone, geranylacetone, vanillin, beta-naphthylmethylether and cedarwood oil are used.

JP 2003113392 describes a composition to be used as an antiperspirant to mask body odor. For the fragrancing, by way of example alpha-pines, lemons, ethyl acetate, cineol, acetylcedrene, geraniol, thymol, tetradecanol, amylsalicylate, vanilla, eucalyptus oil, jasmine oil and spearmint oil are to be used.

WO 03/061609 describes deo preparations containing selected fragrances, which by cross-adaptation can conceal male or female body odor. The numerous substances mentioned there include “menthyl acetate rf” (4-menthyl-3-yl-acetate), Isoraldein® 70 and Rosaphen™.

The object of the present invention was therefore to specify substances or mixtures of substances which in particular for the user, but also third parties, via the nose, are able to provide a perceptible, preferably improved, sense of hygiene and cleanliness, wherein these substances themselves should have a comparatively weak inherent smell, so that it can be combined with the largest possible number of odor types. Here the sense of hygiene and/or cleanliness should preferably occur independently of the odor type used and the effect of the substances used should preferably not be adversely affected by other substances, typically those contained in cosmetic preparations. In particular it should be possible to generate the sense of hygiene and cleanliness in connection with deodorants or antiperspirants.

Surprisingly these desired effects can be achieved by the use of the accords according to the invention described in the following.

Accordingly according to the invention the abovementioned object is achieved by an accord comprising or (essentially) consisting of:

i) ethylene brassylate;
ii) alpha-isomethyl ionone;
iii) 2-methyl-5-phenylpentan-1-ol, and
iv) menthyl acetate,
as well as possible one, two, three, four or all of components v) to ix);
v) alpha-n-methyl ionone;
vi) isomenthyl acetate;
vii) vanillin;
viii) neomenthyl acetate;
ix) neoisomenthyl acetate.

Furthermore, the invention concerns the use of an accord according to the invention to generate a sense of hygiene and/or cleanliness.

None of the above publications indicates that the accords according those inventions are capable of improved combating of body odor or providing an improved sense of hygiene and/or cleanliness. The synergic interactions between the fragrances i) to iv) present in the accord according to the invention [as well as the optionally present fragrances v) to ix], which lead to a lower olfactory perception of body odor, cannot be deduced from the state of the art.

In the context of this text a sense of hygiene and/or cleanliness means a perception by humans via the nasal cavity, in particular via the sense of smell, which is associated with hygiene/cleanliness.

In the context of this text, a sense of hygiene and/or cleanliness alternatively or additionally to that stated above also comprises a suppressed or at least weakened perception of odors with a negative connotation, in particular human body odors. A sense of hygiene and/or cleanliness can accordingly and in the context of this text in particular occur through the lowering of the perceptibility of body odor to a level at which it is no longer noticeable.

The accords according to the invention have as a mixture in our trials demonstrated that in combination with a variety of odor types they are capable of generating a sense of hygiene and/or cleanliness not only in the user but also in third parties. They proved to be effective in neutralizing and/or masking odors, in particular body odor, and more specifically the smell of human sweat, especially the smell of underarm sweat.

### Essential and Optional Components of an Accord According to the Invention in Detail:

i) ethylene brassylate [CAS-No. 105-95-3], ethylene-1,12-dodecanedioate;
ii) alpha-isomethyl ionone [CAS-No. 127-51-5], 4-(2,6,6-trimethyl-2-cyclohexen-1-yl)-3-methyl-3-buten-2-one, also known as gamma-methyl ionone, is available commercially, e.g. under the name Isoraldein® 95 from Givaudan. Here the content of alpha-isomethyl ionone is approximately 85% by weight;
iii) 2-methyl-5-phenylpentan-1-ol [CAS-No. 25634-93-9] (commercially available under the name Rosaphen™ from Symrise);
iv) menthyl acetate [CAS-No. 29066-34-0; (−)-isomer: CAS-No. 2623-23-6], preference is for the acetates of the menthol [=(1α,2β,5α)-Menthol], preferably of the specific d-menthol, of the specific l-menthol and mixtures of the acetates of the racemic specific menthol, with preference for menthyl acetate from racemic menthol;
v) alpha-n-methyl ionone [CAS-No. 127-42-4; CAS-No. 1335-46-2]; 1-(2,6,6-trimethyl-2-cyclohexen-1-yl)-1-penten-3-one, also known as alpha-methyl ionone;
vi) isomenthyl acetate [CAS-No. 20777-45-1], preferably isomenthyl acetate from racemic isomenthol;
vii) vanillin [CAS-No. 121-33-5], here of course natural and/or synthetic vanillin can be used;
iv) neomenthyl acetate [CAS-No. 2230-87-7; (+)-Isomer: CAS-No. 2552-91-2], preferably neomenthyl acetate from racemic neomenthol;
ix) neoisomenthyl acetate, preferably neoisomenthyl acetate from racemic neoisomenthol.

Mixtures comprising components ii) and v) are commercially available, for example under the name Isoraldein® 70 from Givaudan. Isoraldein® 70 contains approximately 60% by weight alpha-isomethyl ionone and approximately 20% by weight alpha-n-methyl ionone.

Preference is for accords according to the invention,
in which the ratio of the weight of i) ethylene brassylate to the total mass of components ii)-iv) is at least 40:100, more preferably at least 50:100;
   and/or
in which the ratio of the weight of iii) 2-methyl-5-phenylpentan-1-ol to the total mass of components i), ii) and iv) is at least 15:100, more preferably 20:100;
   and/or
in which the ratio of the weight of iv) menthyl acetate to the total mass of components i)-iii) is at least 15:100, more preferably at least 20:100.

Further preference is for accords according to the invention comprising or (essentially) consisting of:

i) ethylene brassylate;
ii) alpha-isomethyl ionone;
v) alpha-n-methyl ionone;
iii) 2-methyl-5-phenylpentan-1-ol;
iv) menthyl acetate;
viii) vanillin;
and one, two or all of components vi), vii) and/or ix):
vi) isomenthyl acetate;
viii) neomenthyl acetate;
ix) neoisomenthyl acetate,
wherein the ratio of the weight of i) ethylene brassylate to the total mass of components ii)-v) is at least 40:100, more preferably at least 50:100.

Here the ratio of the weight of the components ii)) alpha-isomethyl ionone and v) alpha-n-methyl ionone is preferably in the range 20:1 to 1:1, more preferably in the range 8:1 to 2:1.

Greatest preference is for accords according to the invention, comprising or (essentially) consist of all components i) to ix) since these accords demonstrated the most distinct odor neutralizing and odor masking effect and allowed the strongest and fastest sense of hygiene and cleanliness to be provided.

In connection with an accord according to the invention “essentially comprising” means that the sum of components i) to ix) is at least 95% by weight, more preferably at least 98% by weight, with reference to the total mass of the accord.

In turn, of the most preferred accords preference is for those which have the abovementioned (and preferably the preferred) ratio of weights for components i) ethylene brassylate, iii) 2-methyl-5-phenylpentan-1-ol and/or iv) menthyl acetate and
in which the ratio of the weight of ii) alpha-isomethyl ionone and v) alpha-n-methyl ionone combined to the total mass of components i), iii), iv) and vi) to ix) is at least 5:100;
   and/or
in which the ratio of the weight of vi) isomenthyl acetate to the mass of component iv) methyl acetate is at least 5:100, more preferably 12:100;
   and/or
in which the ratio of the weight of vii) vanillin to the total mass of components i)-vi) is at least 0.2:100, more preferably 0.4:100 and at the same time preferably a maximum of 1.5:100;
   and/or
in which the ratio of the weight of viii) neomenthyl acetate and ix) neoisomenthyl acetate combined to the mass of component iv) methyl acetate is at least 2:100, more preferably at least 3:100.

A number of especially preferred accords according to the invention are mentioned in the examples below.

A feature of the invention is also a mixture comprising an accord according to the invention and a further fragrance.

The ratio of substances in an accord according to the invention or a preferred accord according to the invention is preferably set so that upon contact of the accord with a body odor or a source of body odor one, several or all the components of the accord

a) interact with the body odor or the source of the body odor so that the inherent olfactory effect of the body odor is weakened and/or
b) interact with a fragrance (preferably a fragrance contained in the accord) in such a way that the inherent olfactory effect of the fragrance, in particular in relation to its capability to mask body odor, is strengthened.

Further preference is for an application according to the invention, in which the sense of hygiene and/or cleanliness is generated by the interaction of the accord according to the invention

(a) with a body odor, wherein the inherent olfactory effect of the body odor is weakened and/or
(b) with a fragrance, wherein the inherent olfactory effect of the fragrance, in particular in relation to its capability to mask body odor, is strengthened.

In the event that in the latter application according to the invention variant (a) applies, the body odor is preferably the smell of human sweat, in particular the smell of underarm sweat.

“Olfactory effect” in this connection means the sensations perceived via the nasal cavity, in particular via the sense of smell.

Body odor, in particular body odor from pathological sweating (hyperhydrosis), can have considerable, including psychological, effects on the individual concerned. Therefore the application according to the invention is also suited to use in the area of medicine, in particular for the prevention, reduction or treatment of the psychological effects of body odor, in particular pathological body odor.

Because of their comparatively neutral odor profile the accords according to the invention can be combined exceptionally well, and easily blended, with other fragrances to form various types of scent and thus scent compositions according to the invention (mixtures according to the invention, but also perfume oils according to the invention, see below), without this resulting in a problematical alteration to the sensorial characteristics of said fragrances. Such scent compositions preferably comprise two, three, four, five, six, seven, eight, nine, ten or more of the fragrances mentioned.

Perfume oils according to the invention or cosmetic preparations according to the invention contain an accord according to the invention or a mixture according to the invention to achieve a sense of hygiene and cleanliness with preferably several scents, in order to make the odor profile of the perfume oil according to the invention or the cosmetic preparation according to the invention more hedonistically pleasant.

Where perfume oils or preparations according to the invention contain, in addition to an accord according to the invention, further fragrances as natural raw materials, in particular essential oils, concretes, absolutes, resins, resinoids, balsams and/or tinctures, these are preferably selected from the group comprising:

ambergris tincture; amyris oil; angelica seed oil; angelica root oil; aniseed oil; valerian oil; basil oil; tree moss absolute; bay oil; armoise oil; benzoe resinoid; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oakmoss absolute; elemi oil; estragon oil; eucalyptus citriodora oil; eucalyptus oil (cineol type); fennel oil; fir needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calamus oil; blue chamomile oil; Roman chamomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemon-grass oil; lovage oil; lime oil distilled; lime oil expressed; linaloe oil; Litsea cubeba oil; laurel leaf oil; mace oil; marjoram oil; mandarin oil; massoi (bark) oil; mimosa absolute; ambrette seed oil; musk tincture; clary sage oil; nutmeg oil; myrrh absolute; myrrh oil; myrtle oil; clove leaf oil; clove bud oil; neroli oil; olibanum absolute; olibanum oil; opopanax oil; orange flower absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil: spike-lavender oil; star anise oil; storax oil; tagetes oil; fir needle oil; tea tree oil; turpentine oil; thyme oil; Tolu balsam; tonka bean absolute; tuberose absolute; vanilla extract; violet leaf absolute; verbena oil; vetiver oil; juniperberry oil; wine lees oil; wormwood oil; wintergreen oil; ylang-ylang oil; hyssop oil; civet absolute; cinnamon leaf oil; cinnamon bark oil.

Individual fragrances are preferably selected from the group comprising:

hydrocarbons, such as for example 3-carene; α-pinene; β-pinene; α-terpinene; γ-terpinene; p-cymene; bisabolene; camphene; caryophyllene; cedrene; farnesene; limonen; longifolene; myrcene; ocimene; valencene; (E, Z)-1,3,5-undecatriene; styrene; diphenylmethane;
aliphatic alcohols, such as for example hexanol; octanol; 3-octanol; 2,6-dimethylheptanol; 2-methyl-2-heptanol, 2-methyl-2-octanol; (E)-2-hexenol; (E)- and (Z)-3-hexenol; 1-octen-3-ol; a mixture of 3,4,5,6,6-pentamethyl-3/4-hepten-2-ol and 3,5,6,6-tetramethyl-4-methyleneheptan-2-ol; (E, Z)-2,6-nonadienol; 3,7-dimethyl-7-methoxyoctan-2-ol; 9-decenol; 10-undecenol; 4-methyl-3-decen-5-ol;
aliphatic aldehydes and their acetals such as for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal-diethylacetal; 1,1-dimethoxy-2,2,5-trimethyl-4-hexene; citronellyl oxyace-aldehyde; 1-(1-methoxy-propoxy)-(E/Z)-3-hexene;
aliphatic ketones and oximes thereof, such as for example 2-heptanone; 2-octanone; 3-octanone; 2-nonanone; 5-methyl-3-heptanone; 5-methyl-3-heptanone oxime; 2,4,4,7-tetramethyl-6-octen-3-one;
aliphatic sulphur-containing compounds, such as for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
aliphatic nitriles, such as for example 2-nonenenitrile; 2-undecenenitrile; 2-tridecenenitrile; 3,12-tridecenenitrtile; 3,7-dimethyl-2,6-octadienenitrile; 3,7-dimethyl-6-octenenitrile;
aliphatic carboxylic acids esters, such as for example (E)- and (Z)-3-hexenylformate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexylbutyrate; (E)- and (Z)-3-hexenyl isobutyrate; hexyl crotonate; ethylisovalerate; ethyl-2-methyl pentanoate; ethyl hexanoate; allyl hexanoate; ethyl heptanoate; allyl heptanoate; ethyl octanoate; ethyl-(E, Z)-2,4-decadienoate; methyl-2-octinate; methyl-2-noninate; allyl-2-isoamyl oxyacetate; methyl-3,7-dimethyl-2,6-octadienoate; 4-Methyl-2-pentyl-crotonate;
acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5,7-octatrien-1-ol; as well as formates, acetates, propionates, isobutyrates, butyrates, isovalerates, pentanoates, hexanoates, crotonates, tiglinates and 3-methyl-2-butenoates thereof;
acyclic terpene aldehydes and ketones, such as, for example, geranial; neral; citronellal; 7-hydroxy-3,7-dimethyloctanal; 7-methoxy-3,7-dimethyloctanal; 2,6,10-trimethyl-9-undecenal; geranylacetone; as well as the dimethyl- and diethylacetals of geranial, neral and 7-hydroxy-3,7-dimethyloctanal;
cyclic terpene alcohols, such as, for example, menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates and 3-methyl-2-butenoates thereof; menthyl formiate; menthyl propionate; methyl butyrate; methyl isobutyrate, methyl isovalerate, menthyl hexanoate; menthyl crotonate, manethyl tiglinate;
cyclic terpene aldehydes and ketones, such as, for example, menthone; isomenthone; 8-mercaptomenthan-3-one; carvone; camphor; fenchone; alpha-ionone; beta-ionone; beta-n-methyl ionone; beta-isomethyl ionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (cedryl methyl ketone);
cyclic alcohols, such as, for example, 4-tert.-butylcyclohexanol; 3,3,5-trimethylcyclohexanol; 3-isocamphylcyclohexanol; 2,6,9-trimethyl-Z2, Z5, E9-cyclododecatriene-1-ol; 2-isobutyl-4-methyltetrahydro-2H-pyran-4-ol;
cycloaliphatic alcohols, such as, for example, alpha,3,3-trimethylcyclo-hexylmethanol; 1-(4-isopropylcyclohexyl)ethanol; 2-methyl-4-(2,2,3-trimethyl-3-cyclopent-1-yl) butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl) hexan-3-ol;
cyclic and cycloaliphatic ethers, such as, for example, cineole; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyl-dodecahydronaphtho[2,1-b]furan; 1,5,9-trimethyl-13-oxabicyclo[10.1.0]-trideca-4,8-diene; rose oxide; 2-(2,4-dimethyl-3-cyclohexen-1-yl)-5-methyl-5-(1-methylpropyl)-1,3-dioxan;
cyclic and macrocyclic ketones, such as, for example, 4-tert.-butylcyclohexanone; 2,2,5-trimethyl-5-pentylcyclopentanone; 2-heptylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;
cycloaliphatic aldehydes, such as, for example, 2,4-dimethyl-3-cyclohexene carbaldehyde; 2-methyl-4-(2,2,6-trimethyl-cyclohexen-1-yl)-2-butenal; 4-(4-hydroxy-4-methylpentyl)-3-cyclohexene carbaldehyde; 4-(4-methyl-3-penten-1-yl)-3-cyclohexene carbaldehyde;
cycloaliphatic ketones, such as, for example, 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert.-butyl-(2,4-dimethyl-3-cyclohexen-1-yl) ketone;
esters of cyclic alcohols, such as, for example, 2-tert.-butylcyclohexyl acetate; 4-tert.-butylcyclohexyl acetate; 2-tert.-pentylcyclohexyl acetate; 4-tert.-pentylcyclohexyl acetate; 3,3,5-trimethylcyclohexyl acetate; decahydro-2-naphthyl acetate; 2-cyclopentylcyclopentylcrotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl-isobutyrate; 4,7-methanooctahydro-5 or 6-indenyl acetate;
esters of cycloaliphatic alcohols, such as, for example, 1-cyclohexylethylcrotonate;
esters of cycloaliphatic carboxylic acids, such as, for example, allyl 3-cyclohexyl-propionate; allyl cyclohexyl oxyacetate; cis- and transmethyl dihydrojasmonate; cis and transmethyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
araliphatic alcohols, such as, for example, benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 3-methyl-5-phenylpentanol; 3-phenyl-2-propen-1-ol; 4-methoxybenzyl alcohol; 1-(4-isopropylphenyl)ethanol;
esters of araliphatic alcohols and aliphatic carboxylic acids, such as, for example, benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha, alpha-dimethylphenylethyl acetate; alpha, alpha-dimethylphenylethyl butyrate; cinnamyl acetate; 2-phenoxyethyl isobutyrate; 4-methoxybenzyl acetate;
araliphatic ethers, such as for example 2-phenylethyl methyl ether; 2-phenylethyl isoamyl ether; 2-phenylethyl-1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a, 5,9b-tetrahydroindeno[1,2-d]-m-dioxin; 4,4a,5,9b-tetrahydro-2,4-dimethylindeno[1,2-d]-m-dioxin;
aromatic and araliphatic aldehydes, such as, for example, benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl) propanal; 2-methyl-3-(4-tert.-butylphenyl) propanal; 2-methyl-3-(4-isobutylphenyl)propanol; 3-(4-tert.-butylphenyl) propanal; cinnamaldehyde; alpha-butylcinnamaldehyde; alpha-amylcinnamaldehyde; alpha-hexylcinnamaldehyde; 3-methyl-5-phenylpentanol; 4-methoxybenzaldehyde; 4-hydroxy-3-ethoxybenzaldehyde; 3,4-methylene-dioxybenzaldehyde; 3,4-dimethoxybenzaldehyde; 2-methyl-3-(4-methoxyphenyl) propanal; 2-methyl-3-(4-methylenedioxyphenyl) propanal;
aromatic and araliphatic ketones, such as, for example, acetophenone; 4-methylacetophenone; 4-methoxyacetophenone; 4-tert.-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tert.-butyl-1,1-dimethyl-4-indanyl methyl ketone; 1-[2,3-dihydro-1,1,2,6-tetramethyl-3-(1-methyl-ethyl)-1H-5-indenyl]ethanone; 5′, 6′, 7′,8′-tetrahydro-3′, 5′, 5′, 6′, 8′,8′-hexa-methyl-2-acetonaphthone;
aromatic and araliphatic carboxylic acids and esters thereof, such as, for example, benzoic acid; phenylacetic acid; methyl benzoate; ethyl benzoate; hexyl benzoate; benzyl benzoate; methyl phenyl acetate; ethyl phenyl acetate; geranyl phenyl acetate; phenylethyl phenyl acetate; methyl cinnamate; ethyl cinnamate; benzyl cinnamate; phenylethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
nitrogen-containing aromatic compounds, such as, for example, 2,4,6-trinitro-1,3-dimethyl-5-tert.-butylbenzene; 3,5-dinitro-2,6-dimethyl-4-tert.-butylacetophenone; cinnamonitrile; 5-phenyl-3-methyl-2-pentenonitrile; 5-phenyl-3-methylpentanonitrile; methyl anthranilate; methyl-N-methylanthranilate;

Schiffs bases of methyl anthranilate with 7-hydroxy-3,7-dimethyloctanal, 2-methyl-3-(4-tert.-butylphenyl) propanal or 2,4-dimethyl-3-cyclohexene carbaldehyde; 6-isopropylquinoline; 6-isobutylquinoline; 6-sec.-butylquinoline; indole; skatole; 2-methoxy-3-isopropylpyrazine; 2-isobutyl-3-methoxypyrazine; phenols, phenyl ethers and phenyl esters, such as, for example, estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenyl acetate;

heterocyclic compounds, such as, for example, 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones, such as, for example, 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene-1,13-tridecanedioate; coumarin; 2,3-dihydrocoumarin; octahydrocoumarin.

It has also been discovered that perfume oils according to the invention comprising (i) an accord according to the invention or a mixture according to the invention and (ii) one or more of the fragrances of group (B) lead to an overall improvement in the sense of hygiene and cleanliness with improved odor neutralising/odor masking effect.

In a further preferred embodiment the accord according to the invention or the mixtures according to the invention are combined with, preferably, two, three, four, five or more of the fragrances from group (B) mentioned below, since such combinations can give a particularly distinct sense of hygiene and cleanliness, wherein they demonstrate an improved odor neutralizing/odor masking effect, in particular in relation to the capability to mask body odor.

Group (B) of these fragrances preferably to be combined with an accord according to the invention comprises: (here in some cases the normal industrial product names and registered trademarks of various firms are given)

alpha-hexylcinnamaldehyde, 2-phenoxyethylisobutyrate (Phenirat), dihydromyrcenol (2,6-dimethyl-7-octen-2-ol), methyl dihydrojasmonate; preferably with a content of cis-isomers of >60 by weight (Hedione, Hedione HC), 4,6,6,7,8,8-hexamethyl-1,3,4,6,7,8-hexahydrocyclopenta[g]benzopyran (Galaxolid), tetrahydrolinalool (3,7-dimethyloctan-3-ol), ethyllinalool, benzylsalicylate, 2-methyl-3-(4-tert-butyl-phenyl)propanal (Lilial), cinnamon alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat), citronellol, linalyl acetate, styrolyl acetate(1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super), hexylsalicylate, 4-tert.-butylcyclohexyl acetate (Oryclon), 2-tert.-butylcyclohexyl acetate (Agrumex HC), alpha-ionone (4-(2,2,6-trimethyl-2-cyclohexen-1-yl)-3-buten-2-one), coumarin, terpinyl acetate, 4-(4-hydroxy-4-methylpentyl)-3-cyclohexene carboxaldehyde (Lyral), alpha-amyl cinnamon aldehyde, (E)- and/or (Z)-3-methylcyclopentadec-5-enone (Muscenon), 15-pentadec-11-enolide and/or 15-pentadec-12-enolide (Globalide), 15-cyclopentadecanolide (Macrolide), 1-(5,6,7,8-tetrahydro-3,5,5,6,8,8-hexamethyl-2-naphthalenyl)ethanone (Tonalid), 2-isobutyl-4-methyltetrahydro-2H-pyran-4-ol (Florol), 2-ethyl-4-(2,2,3-trimethyl-3-cyclopenten-1-yl)-2-buten-1-ol (Sandolen), menthol (preferably l-menthol or racemic menthol, with particular preference for l-menthol), anethole, geraniol, nerol, linalool, citronellol, linalyl acetate, 2-phenylethyl alcohol, 2,2-dimethyl-3-(3-methylphenyl)-propanol (Majantol), rose oxide (4-methyl-2-(2-methyl-1-propenyl)tetrahydropyran), allyl heptanoate, 4-methylacetophenone, (4aR,5R,7aS,9R)-octahydro-2,2,5,8,8,9a-hexamethyl-4H-4a,9-methanoazuleno(5,6-d)-1,3-dioxol) (Ambrocenide), Timberol (1-(2,2,6-trimethylcyclohexyl)hexan-3-ol), Floropal (2,4,6-trimethyl-4-phenyl-1,3-dioxan), benzylacetone, methyl cinnamate, 3a,6,6,9a-tetramethyldodecahydronaphtho[2,1-b]furan (Ambroxid).

In perfume oils and cosmetic preparations according to the invention, in particular (deo) preparations, the ratio of the weight of total quantity of the accord according to the invention to the total quantity of the fragrance from group (B) is preferably in the range 10:1 to 1:50, preferably in the range 5:1 to 1:25.

Preferred preparations according to the invention include in particular for a further improved effectiveness of the accord according to the invention, in particular from deo preparations, one or more yielding agents from the group (H) comprising dipropylene glycol (DPG), diethylphthalate (DEP), triethylcitrate (TEC), isopropylmyristate (IPM), and/or benzylbenzoate (BB).

Individual preferred cooling agents for use in connection with the present invention (in perfume oils or preparations according to the invention) are listed in the following. The listed cooling agents can also be used in combination with each other here: menthonglycerinacetal (trade name: Frescolat® MGA), menthyllactate (trade name: Frescolat® ML, this is preferably for menthyllactate a case of l-menthyllactate, in particular l-menthyl-l-lactate), substituted menthyl-3-carboxylic acid amide (e.g. menthyl-3-carboxylic acid-N-ethylamide), 2-isopropyl-N-2,3-trimethylbutanamide, substituted cyclohexane carboxylic acid amide, 3-menthoxypropane-1,2-diol, 2-hydroxyethylmenthylcarbonate, 2-hydroxypropylmenthylcarbonate, N-acetyl glycine menthyl ester, Isopulegol, menthyl hydroxycarboxylic acid ester (e.g. menthyl-3-hydroxybutyrate), monomenthylsuccinate, 2-mercaptocyclodecanone, menthyl-2-pyrrolidin-5-one carboxylate, 2,3-dihydroxy-p-menthane, 3,3,5-trimethyl cyclohexanone glycerine ketal, 3-menthyl-3,6-di- and -trioxalkanoate, 3-menthylmethoxy acetate, Icilin.

Preferred cooling agents are: menthone glycerine acetal (trade name: Frescolat® MGA), menthyl lactate (preferably 1-menthyl lactate, in particular l-menthyl-l-lactat, trade name: Frescolat® ML), 3-menthoxy propane-1,2-diol, 2-hydroxyethyl menthyl carbonate, 2-hydroxypropyl menthyl carbonate, particular preference being for l-menthyl-l-lactate.

Part of the invention is also a perfume oil for generating a sense of hygiene and cleanliness, comprising an accord according to the invention.

The accords according to the invention and the perfume oils containing an accord according to the invention have in particular an odor neutralizing/odor masking effect on body odor, in particular on the smell of (underarm) sweat.

An important advantage of the perfume oils according to the invention is that the accords according to the invention, because of their—for fragrances—comparatively weak inherent odor only contribute slightly to the overall odor impression.

In a perfume oil according to the invention the total mass of accord according to the invention is preferably in the range 5 to 50% by weight, more preferably in the range 10 to 40% by weight, with particular preference for a range of 15 to 30% by weight, with reference to the total mass of the perfume oil.

A feature of the invention is also cosmetic preparations which comprise a perfume oil according to the invention, a mixture according to the invention or an accord according to the invention. Preferred cosmetic preparations according to the invention include a perfume oil according to the invention or an accord according to the invention in a quantity in the range of 0.1 to 3% by weight, preferably in the range of 0.2 to 2% by weight, with particular preference for a range of 0.3 to 1.5% by weight, with reference to the total mass of the cosmetic preparation.

In the manufacture of a preparation according to the invention an accord according to the invention, a mixture according to the invention or a perfume oil containing an accord according to the invention can be mixed in the normal way that will be familiar to a person skilled in the art with the other components of the preparation. In a preferred embodiment initially an accord according to the invention or a mixture according to the invention is introduced in to the basic formulation of the cosmetic preparation concerned, in particular a (deo-) preparation, and then a perfume oil according to or not according to the invention is introduced into this basic formulation containing the accord according to the invention.

Like the perfume oils according to the invention, the cosmetic preparations according to the invention have the advantage that because of the comparatively weak inherent smell of the accords according to the invention the cosmetic preparations according to the invention are not associated with specific odor types and thus as regards their actual composition are restricted only very slightly, if at all, by the accords according to the invention. With such cosmetic preparations it is preferable for a perceptible sense of hygiene and cleanliness to develop quickly. It may, however, be desirable that the effect of the accords according to the invention is delayed or, for example, only occurs when the body starts to sweat. This can be achieved through appropriate measures when incorporating the corresponding accords according to the invention into the perfume oils according to the invention or cosmetic preparations (see also below).

The accords, mixtures and perfume oils according to the invention can be included in all products, in particular (deo-)preparations (deodorants), which are suitable for combating and/or masking the unpleasant smell of sweat, with products that remain on the skin or hair (so-called leave-on products) being particularly advantageous.

The form of administration of the leave-on products can vary greatly and may include sprays, gels, foams, balms, tinctures, lotions, crèmes, milks, oils and sticks. The following products can be cited as examples: deodorants (e.g. pump sprays, atomizers, propellant aerosol sprays, roll-ons, gels, crèmes, sticks, foams); air fresheners (e.g. gels or liquids); body lotions, body crèmes or body milks (emulsions or micro-emulsions of the O/W or W/O type, or also multiple emulsions such as O/W/O), body oils, foot crèmes, hair setting lotions, hairsprays or alcohol or alcohol-water solutions for application to the skin.

Preferred deodorants according to the invention come in an application form selected from the group comprising sticks, crèmes, gels, lotions, foams, roll-on preparations (e.g. roll-on gels, roll-on emulsions), powder sprays, aerosol or non-aerosol sprays (pump sprays).

Preference is for sticks, roll-on preparations and sprays, of which, in turn, particular preference is for aerosol sprays, since these allow the preparations containing accords or perfume oils according to the invention to provide the strongest and most effective sense of hygiene and cleanliness, with them demonstrating an excellent odor neutralizing and odor masking effect against the smell of sweat.

Accordingly particularly preferred cosmetic preparations according to the invention are deodorants and/or antiperspirants.

The cosmetic deodorants and antiperspirants mixed with accords or mixtures according to the invention or with perfume oils according to the invention can, depending on the way in which they work, contain one or more of the following active substances:

### 1.) Deodorants

1-1.) Antimicrobial acting substances, which inhibit the development of the micro-organisms responsible for the smell of sweat. Examples of these are Triclosan® (5-chloro-2-(2,4-dichlorophenoxy)phenol), triclocarban, chlorhexidine, chlorhexidine hydrochloride, chlorhexidine gluconate, 2-phenoxy ethanol, farnesol, glycerine esters and ethers such as glycerine monolaurate, glycerine monocaprinate, hexoxyglycerine, octoxyglycerine (=ethylhexylglycerine, 3-(2-ethylhexyloxy-1,2-propandiol), e.g. Sensiva® SC 50 from Schülke & Mayr), aliphatic 1,2-dioles such as 1,2-decandiole (EP 1 269 983), araliphatic alcohols such as those described in EP 799 174, preferably 4-methyl-4-phenyl-2-pentanol (Vetikol; WO 03/024907) or 2-methyl-4-phenyl-2-butanol (1,1-dimethyl-3-phenylpropanol, alpha,alpha-dimethylphenethylcarbinol), 1-menthylmethylether as described in WO 02/41861, 2-benzylheptan-1-ol (jasmol; 2-n-pentyl-3-phenylpropan-1-ol), 2,2-dimethyl-3-phenylpropanol (muguet alcohol; c.f. U.S. Pat. No. 4,091,090), antimicrobially-acting secondary alcohols, such as those described in WO 2005/004601, in particular 3-methyl-6-phenyl-2-hexanol, 4-(2,4-dimethylphenyl)-2-butanol, 6-(4-isopropylphenyl)-3-methyl-2-hexanol, 4-(2,4,5-trimethylphenyl)-2-butanol, 3,3-dimethyl-4-phenyl-2-butanol, 3-methyl-4-(2-methylphenyl)-2-butanol, 3-methyl 4-(2,4,5-trimethylphenyl)-2-butanol, 3,3-dimethyl-4-(2,5-dimethylphenyl)-2-butanol, 3-methyl-4-(4-methylphenyl)-2-butanol, 4-(3,4-dimethylphenyl)-3-methyl-2-butanol, 4-(3,4-dimethylphenyl)-2-butanol, 4-(2,4,6-trimethylphenyl)-2-butanol, 4-bicyclo[2.2.1]hept-2-yl-2-butanol, 3-methyl-4-(2,4,6-trimethylphenyl)-2-butanol, 4-(2,6,6-trimethylbicyclo[3.1.1]hept-3-yl)-2-butanol, 5,5-dimethyl-6-(3-methylphenyl)-hexan-2-ol, 4-bicyclo[2.2.1]hept-2-yl-3-methyl-2-butanol, 3,3-dimethyl-4-(2,4,6-trimethylphenyl)-2-butanol, 4-(4-t-butylphenyl)-3-methyl-2-butanol, 4-(4-isopropylphenyl)-3-methyl-2-butanol, 6-(3,4-dimethylphenyl)-3-methyl-2-hexanol, 4-(2,4-dimethylphenyl)-3-methyl-2-butanol, 6-(3,4-dimethylphenyl)-2-hexanol, aliphatic carboxylic acids such as 2-hexyloctanic acid, 2-hexyldecanic acid, 2-butyloctanic acid or 2-butyldecanic acid.
1-2.) Enzyme-inhibiting substances, which suppress the effect of enzymes involved in the formation of the smell of sweat. Examples of this are citric acid esters and metal-chelating substances such as EDTA (ethylene diamine tetra-acetic acid), EGTA (ethylene glycol tetra acetic acid) and DTPA (diethylene triamine pentaacetic acid-pentetic acid).
1-3.) Odor-absorbers that absorb the substances responsible for the smell of sweat. An example of this is zinc ricinoleate.
1-4.) Odor-Neutralizing Substances, which Due to their Special Characteristics, are Able to Reduce Olfactory Perception.
1-5.) Odor-masking substances, which due to their strong odor and their odor type are able to mask the smell of sweat. Examples are quoted in, for example WO 2006/124230. In anhydrous cosmetic preparations, these substances can be used in spray-dried or encapsulated form, e.g. using cyclodextrin.

### 2.) Antiperspirants

Antiperspirants inhibit the secretion of sweat and thus remove the bacteria responsible for body odor from the breeding ground. As antiperspirants astringent metal salts are generally used, in particular inorganic and organic metal salts of the elements aluminum, zinc, magnesium, tin and zirconium as well as mixtures of these, wherein in particular halogenides such as aluminum hydroxychloride, zirconyl oxychloride and zirconyl hydroxychloride as well as mixtures of these are used. Frequently these aluminum and zirconium salts and their mixtures are also used in complex form, with propylene glycol, polyethylene glycol or glycerine being used as complexing agents.

Here the following antiperspirants or mixtures of these are preferred:

aluminium chlorohydrate; aluminium sesquichlorohydrate, aluminium chlorohydrex PG, aluminium dichlorohydrex PG, aluminium sesquichlorohydrex PG, aluminium chlorohydrex PEG, aluminium dichlorohydrex PEG, aluminium sesquichlorohydrex PEG, aluminium chloride (preferably in the form of a 15% by weight (aqueous) solution), aluminium zirconium chlorohydrate, aluminium zirconium trichlorohydrate, aluminium zirconium tetrachlorohydrate, aluminium zirconium pentachlorohydrate, aluminium zirconium octachlorohydrate, aluminium zirconium trichlorohydrex-gly, aluminium zirconium tetrachlorohydrex-gly, aluminium zirconium pentachlorohydrex-gly, aluminium zirconium octachlorohydrex-gly, buffered aluminium sulphate, basic aluminium chloride, zirconium hydroxychloride, zirconium chloride, basic aluminium nitrate, basic aluminium chloride combined with zirconyloxychloride and -hydroxychloride, organic complexes of basic aluminium chlorides and/or zirconium chloride and/or zirconium hydroxychloride.

In turn, preference here is for aluminum or aluminum zirconium complexes with a metal/anion ratio in the range 0.9:1 to 2.1:1, wherein the anion is preferably selected from the group comprising Cl⁻, Br⁻, I⁻ and/or NO₃⁻, if necessary in combination with additives such as amino acids (preferably glycine) or mono- or polyvalent alcohols. The polyvalent alcohols are preferably di-, tri- or polyols with 3-12 C-atoms, with preference here being in turn for glycerine, propylene glycol, diglycerine, tripropylene glycol, sorbitol, 1,2,4-butanetriol or 1,2,6-hexanetriol and mixtures thereof, with particular preference for glycerine and diglycerine. Advantageous monoalcohols are glycol ethers such as monoalkylether or alpha-hydroxy acids such as lactic acid.

In connection with the present invention mixtures of antiperspirants comprising (i) aluminum and/or zirconium and (ii) zinc and/or tin can be used, such as Al/Zr/Zn, Al/Zn, Al/Sn or Al/Sr/Sn, wherein mixtures comprising one or more antiperspirants from among the abovementioned preferred aluminum- and or zirconium-based antiperspirants are in turn preferred.

If necessary the deodorants and antiperspirants according to the invention, apart from the special active ingredients described above, can contain additional substances:

propellant gases, ethanol, propylene glycol, emulsifiers such as aminomethylpropanol, skin care/moisturising agents such as 2-octyldodecanol, isopropylmyristate, isopropylpalmitate, stearamide, sorbitol, glycerine and modified polyethylene- and polypropylenglycols, vitamins and their derivatives (e.g. tocopherol (vitamin E), tocopheryl acetate (vitamin E-acetate) and ascorbic acid (vitamin C)), panthenol, allantoine, plant extracts, such as Aloe Vera and proteins, lustre agents, electrolyte salts such as KCl, NaCl, gelling substances such as hydroxyalkylcelluloses, fatty alcohols, fatty acids, fatty alcohol fatty acid esters, fatty acid glyceryl esters or similar, liquid carriers and solvents such as volatile and non-volatile silicon oils, solid carriers such as talcum, silica gels, and similar; antioxidants and preservatives, UV-light protective filters, cooling agents, additional fragrancing materials, in order to modify odor types, which increase sensory acceptance and/or improve the hedonistic feeling.

A scent composition according to the invention (a mixture according to the invention or a perfume oil according to the invention) containing an accord according to the invention can however also be added to the cosmetic preparations with deodorant or antiperspirant effect in pure form or, for example, also by means of a carrier or in encapsulated form.

For many applications it is advantageous to use a scent composition according to the invention adsorbed by a carrier containing an accord according to the invention that ensures both the distribution of the fragrances in the product and a controlled release when applied. Such carriers may be porous inorganic materials such as light grade sulphate, silica gels, zeoliths, gypsum, clays, granulated clay, gas concrete and so on, or organic materials such as woods, cellulose-based materials, sugar or plastics such as PVC, polyvinyl acetate or polyurethane.

For other applications it is advantageous to use a scent composition containing an accord according to the invention in micro-encapsulated and spray-dried form, as an inclusion complex or extrusion product and to add the (preliminary) product to be perfumed in this form.

The characteristics of such modified fragrance mixtures (perfume oils) are in many cases optimized by coating with suitable materials to achieve a more targeted scent release, for which preferably waxy plastics such as polyvinyl alcohols are used.

The microencapsulation of the scent composition containing an accord according to the invention can, for example, take place by means of the so-called coacervation method with the help of capsule materials such as polyurethane-like materials or soft gelatine The spray-dried perfume oils according to the invention can, for example, be manufactured by spray drying of an emulsion or dispersion containing the perfume oil, wherein for the carrier modified starches, proteins, dextrin and vegetable gums can be used. Inclusion complexes can, for example, be manufactured by introducing dispersions of the perfume oil and cyclodextrins or urea derivatives into a suitable solvent, such as water. Extrusion products can be created by melting the perfume oils with a suitable waxy substance followed by extrusion and subsequent hardening, if necessary in a suitable solvent, such as isopropanol.

Consistent with that stated above, part of the invention also comprises the use of a mixture according to the invention, a perfume oil according to the invention or a preparation according to the invention, to generate a sense of hygiene and/or cleanliness.

Here the sense of hygiene or cleanliness is preferably generated by the interaction of an accord according to the invention,

(i) with a body odor, wherein the inherent olfactory effect of the body odor is weakened and/or
(ii) with a fragrance, wherein the inherent olfactory effect of the fragrance, in particular in relation to its capability to mask body odor, is strengthened,
wherein the body odor is preferably the smell of human sweat, in particular the smell of underarm sweat.

Consistent with that stated above likewise part of the invention is a method for cosmetically generating a sense of hygiene and/or cleanliness, comprising the following stages:

a) Preparation (i) of an accord according to the invention or
(ii) of a perfume oil according to the invention or
(iii) of a mixture according to the invention or
(iv) of a preparation according to the invention and
   b) Application of the accord according to the invention, the mixture, the perfume oil or the preparation, so that the olfactory effect of the accord according to the invention, the mixture, the perfume oil or the preparation is perceptible enough that a sense of hygiene and/or cleanliness is generated,

In the method according to the invention it is preferable for application to be to the skin.

In many cases a method according to the invention is preferred in which the application takes place in the presence of perceptible body odor. Likewise preferred with the method according to the invention is that the application takes place in the area of a source or a potential source of body odor. A source or potential source can in particular be considered to be the armpits.

In a preferred method according to the invention application is by spraying.

In the following further explanations of the invention, or individual aspects of it, are provided by means of the claims and examples.

### EXAMPLES

Unless otherwise stated all figures refer to the weight. Abbreviations used: DPG: dipropylene glycol, PPG: polypropylene glycol.

### Example 1

### Perfume Oil for Use in Deodorants and Antiperspirants (not According to the Invention)

| | Parts by |
|---|---|
| Components | weight |
| 10-undecenal, 10% in DPG | 2.00 |
| Vertocitral (2,4-dimethyl-3-cyclohexencarboxaldehyde) | 0.50 |
| cis-3-hexenyl acetate, 10% in DPG | 7.50 |
| Allylamylglycolate | 2.00 |
| Melonal (2,6-dimethyl-5-hepten-1-al) | 0.50 |
| Bergamot oil | 70.00 |
| Dihydromyrcenol | 80.00 |
| Cyclogalbanate (allylcyclohexyloxy acetate) | 20.00 |
| Terpinyl acetate | 40.00 |
| Litsea cubeba oil | 2.00 |
| Lemon oil | 50.00 |
| Orange oil | 20.00 |
| Grapefruit oil | 10.00 |
| Lavandin oil abrialis | 10.00 |
| Isobornyl acetate | 3.00 |
| Lilial (2-methyl-3-(4-tert-butyl-phenyl)propanal) | 10.00 |
| Calone 1951 (7-methyl-2H-1,5-benzodioxepin-3(4H)-one) | 2.50 |
| Florhydral (3-(3-isopropylphenyl)butanal) | 1.50 |
| Florol (2-isobutyl-4-methyltetrahydro-2H-pyran-4-ol) | 12.00 |
| Tetrahydrolinalool | 75.00 |
| Geranium oil | 5.00 |
| Isodamascon (1-(2,4,4-trimethyl-2-cyclohexen-1-yl)-2- | 2.00 |
| buten-1-one), 10% in DPG | |
| Resedafol, 10% in DPG [2-(1-propoxyethoxy)ethyl]benzol | 1.00 |
| Methyl dihydrojasmonate (Hedione HC) | 158.00 |
| L-menthylmethylether | 50.00 |
| Jessemal (3-butyl-5-methyl tetrahydropyran-4-yl-acetate) | 4.00 |
| Benzyl salicylate | 10.00 |
| Anethol | 3.00 |
| Methylcedrylketone | 50.00 |
| Iso E Super⁺⁺ | 25.00 |
| Ambrocenide ((4aR,5R,7aS,9R)-octahydro-2,2,5,8,8,9a- | 3.00 |
| hexamethyl-4H-4a,9-methanoazuleno(5,6-d)-1,3-dioxol), | |
| 1% in DPG | |
| Timberol (1-(2,2,6-trimethylcyclohexyl)hexan-3-ol) | 2.00 |
| Patchouli oil | 3.50 |
| Evernyl (methyl-2,4-dihydroxy-3,6-dimethylbenzoate) | 1.50 |
| Labdanum absolute, 20% in DPG | 2.00 |
| Amber Core (1-[[2-(1,1-dimethylethyl)cyclohexyl]oxy]-2- | 10.00 |
| butanol) | |
| Ambraketal (dodecahydro-3,8,8,11a-tetramethyl-5H-3,5a- | 1.50 |
| epoxynaphth[2,1-c]oxepin) | |
| Hydroxyambran (2-cyclododecylpropanol), 50% in DPG | 5.00 |
| Macrolide (15-cyclopentadecanolide) | 35.00 |
| Globalide (15-pentadec-(11/12)-enolide) | 20.00 |
| Globanone ((E/Z)-8-cyclohexadecenone) | 20.00 |
| Isopropylmyristate | 170.00 |
| Total | 1,000.00 |

⁺⁺Octahydro-2,3,8,8-tetramethyl-2-acetonaphthone and 2-acetyl-1,2,3,4,6,7,8-octahydro-2,3,8,8-tetramethylnaphthaline

### Example 2

### Perfume Oil with Rose Odor for Use in Deodorants and Antiperspirants (not According to the Invention)

| | Parts by |
|---|---|
| Components | weight |
| Acetophenone, 10% in DPG | 10.00 |
| n-undecanal | 5.00 |
| Aldehyde C14 so-called (peach aldehyde) | 15.00 |
| Allylamylglycolate, 10% in DPG | 20.00 |
| Amylsalicylate | 25.00 |
| Benzyl acetate | 60.00 |
| Citronellol | 80.00 |
| d-Limonen | 50.00 |
| Trans-9 decenol | 15.00 |
| Dihydromyrcenol | 50.00 |
| Dimethylbenzyl carbinyl acetate | 30.00 |
| Diphenyl oxide | 5.00 |
| Eucalyptus oil | 10.00 |
| Geraniol | 40.00 |
| Nerol | 20.00 |
| Geranium oil | 15.00 |
| Hexenol cis-3, 10% in DPG | 5.00 |
| Hexenyl salicylate cis-3 | 20.00 |
| Indol, 10% in DPG | 10.00 |
| Alpha-Ionone | 15.00 |
| Beta-Ionone | 5.00 |
| Lilial (2-methyl-3-(4-tert-butyl-phenyl)propanal) | 60.00 |
| Linalool | 40.00 |
| Methylphenyl acetate | 10.00 |
| Phenylethyl alcohol | 275.00 |
| Styrene acetate | 20.00 |
| Terpineol | 30.00 |
| Tetrahydrolinalool | 50.00 |
| Cinnamon alcohol | 10.00 |
| Total | 1,000.00 |

### Example 3

### Perfume Oil with White Petal Odor for Use in Deodorants and Antiperspirants (not According to the Invention)

| | Parts by |
|---|---|
| Components | weight |
| Benzyl acetate | 60.00 |
| Citronellyl acetate | 60.00 |
| Cyclamenaldehyde (2-methyl-3-(4-isopropylphenyl)propanal | 20.00 |
| Dipropylene glycol | 60.00 |
| Ethyllinalool | 40.00 |
| Floral (2-isobutyl-4-methyltetrahydro-2H-pyran-4-ol) | 30.00 |
| Globanone ((E/Z)-8-cyclohexadecen-1-one) | 180.00 |
| Hedione (methyl dihydrojasmonate) | 140.00 |
| Hexenyl salicylate, cis-3 | 10.00 |
| Vertocitral (2,4-dimethyl-3-cyclohexencarboxaldehyde) | 5.00 |
| Hydratropaaldehyde, 10% in DPG | 5.00 |
| Isodamascon (1-(2,4,4-trimethyl-2-cyclohexen-1-yl)-2- | 5.00 |
| buten-1-one), 10% in DPG | |
| Isomuscone (Cyclohexadecanone) | 40.00 |
| Jacinthaflor (2-methyl-4-phenyl-1,3-dioxolan) | 10.00 |
| Cis-jasmone, 10% in DPG | 20.00 |
| Linalool | 50.00 |
| Linalyl acetate | 30.00 |
| Methyl benzoate, 10% in DPG | 25.00 |
| Para-methylkresol, 10% in DPG | 10.00 |
| Nerol | 20.00 |
| Phenylpropyl aldehyde | 5.00 |
| Phenylethyl alcohol | 82.00 |
| Tetrahydro geraniol | 13.00 |
| 2,2-dimethyl-3-cyclohexyl-1-propanol | 80.00 |
| Total | 1,000.00 |

### Example 4

### Accords (Acc. A to Acc C) According to the Invention to Achieve a Hygiene and Cleanliness Effect

The following are mixed:

| | | Acc. A | Acc. B | Acc. C |
|---|---|---|---|---|
| | | % by | % by | % by |
| Component(s) | Constituent(s) | weight | weight | weight |
| Ethylene brassylate | i) | 51.20 | 50.00 | 50.00 |
| Isoraldein ® 70* | ii), v) | 8.00 | 8.00 | 8.00 |
| 2-methyl-5- | iii) | 20.00 | 20.00 | 20.00 |
| phenylpentan-1-ol | | | | |
| L-menthyl acetate | iv) | 18.20 | — | — |
| Isomenthyl acetate | vi) | 2.00 | — | — |
| racemic | | | | |
| Acetate of a mixture of | iv), vi), viii), ix) | — | 21.60 | — |
| menthol isomers | | | | |
| Acetate of a mixture if | iv), vi), viii), ix) | — | — | 21.60 |
| menthol isomers** | | | | |
| Vanillin^{#} | vii) | 0.60 | 0.40 | 0.40 |

*Isoraldein ® 70 (Givaudan), contains approximately 60% by weight alpha-isomethyl ionone and 20% by weight alpha-n-methyl ionone.
**Acetate of a mixture of racemic menthol isomers comprising 80.0% by weight rac. menthyl acetate, 14.3% by weight rac. isomenthyl acetate, 1.1% by weight rac. neomenthyl acetate, 1.8% by weight rac. neoisomenthyl acetate and 2.8% by weight paramenthyl acetate (4-isopropyl-3-methylcyclohexyl acetate, not a constituent of iv), vi), viii), ix)).
***: Acetate of a mixture of racemic menthol isomers comprising 86.5% by weight rac. menthyl acetate, 8.2% by weight rac. isomenthyl acetate, 2.2% by weight rac. neomenthyl acetate, 1.2% by weight rac. neoisomenthyl acetate and 1.9% by weight paramenthyl acetate (4-isopropyl-3-methylcyclohexyl acetate, not a constituent of iv), vi), viii), ix)).
^{#}e.g. Catechol.

### Example 5

### Manufacture of an Alcohol-Based Deodorant Spray

In 49 parts by weight of 96% ethanol the each of the following are dissolved:

a) 1 part by weight of the perfume oil manufactured in example 1.
b) 0.8 parts by weight of the perfume oil manufactured in example 1 and 0.2 parts by weight of the accord “Acc. B” manufactured in example 4.

From 1 part by weight of the resultant solution and 1 part by weight of a propellant gas (propane/butane; pressure 2.5 to 2.7 bar) an aerosol spray was produced which was used as a deodorant spray on a large number of test subjects to combat existing odor of underarm sweat.

The evaluation via a trained sensory panel demonstrated that the deodorant containing mixture b), was better at masking the smell of sweat and provided a better sense of hygiene and cleanliness than the deodorant that contained mixture a).

### Example 6

### Production of an Antiperspirant Spray

To a mixture of suitable carriers comprising 12.5 parts by weight Miglyol 840 Gel B (a mixture of propylene glycol dicaprylate-dicaprate, steralkonium hectorite and propylene carbonate; manufacturer: Sasol), and 46.5 parts by weight silicon oils (cyclomethicone, dimethiconol; manufacturer Dow Corning) 40 parts by weight of aluminum hydrochlorate were added. Once the mixture had homogenized, the following were added:

a) 1 part by weight of the perfume oil manufactured in example 1;
b) 0.8 parts by weight of the perfume oil manufactured in example 1 and 0.2 parts by weight of the accord “Acc. C” manufactured in example 4.

From 1 part by weight of the resultant solution and 3 parts by weight of a propellant gas (propane/butane; pressure 2.5 to 2.7 bar) an aerosol spray was produced which was used as an antiperspirant spray on a large number of test subjects to prevent the smell of underarm sweat.

The evaluation by a trained sensory panel showed that antiperspirants containing mixture b), offered better masking of the existing smell of sweat than the corresponding product containing only mixture a). Accordingly the antiperspirant with mixture b) was considered to be much more suitable as regards the generation of a sense of hygiene and cleanliness.

And the test subject on whom the antiperspirants were tested themselves felt when they used the product containing mixture b) significantly cleaner and more hygienic.

### Example 7

### Deo Sticks

| | A | B | C | D |
|---|---|---|---|---|
| | % by | % by | % by | % by |
| Components | weight | weight | weight | weight |
| Sodium stearate | 7.00 | 8.00 | 7.00 | 7.00 |
| Sodium palmitate | 1.00 | — | — | 0.50 |
| 1,2-propylene glycol | 42.00 | 45.00 | 44.00 | 48.00 |
| 1,2-butylene glycol | 3.00 | — | 2.00 | — |
| 1,1-dimethyl-3- | 0.50 | 0.25 | 0.30 | — |
| phenylpropanol | | | | |
| 2-butyloctanic acid | — | 0.50 | 0.10 | 0.20 |
| 2-hexyldecanic acid | 0.10 | — | — | 0.20 |
| Polyethylenglycol (25) | 3.00 | 3.00 | — | — |
| cetearylether | | | | |
| Ethanol | 20.00 | 20.00 | — | — |
| Farnesol | — | 0.25 | 0.50 | — |
| Triclosan ® (5-chloro-2-(2,4- | 0.30 | — | — | 0.30 |
| dichlorphenoxy)phenol) | | | | |
| Parabens (mixture of | 0.30 | — | 0.40 | — |
| methyl-, ethyl-, propyl-, | | | | |
| butyl-, isobutylparaben) | | | | |
| 1,2-hexandiol/1,2-octandiol | — | 0.50 | — | 0.50 |
| (1:1) (Symdiol 68) | | | | |
| 1,2-pentandiol (Hydrolite-5) | 0.50 | — | 2.00 | — |
| (—)-Alpha-Bisabolol, nat. | 0.10 | — | — | 0.12 |
| “Acc. A” according to | 0.20 | — | — | — |
| example 4 | | | | |
| Perfume oil according to | 0.90 | — | — | 0.50 |
| example 1 | | | | |
| “Acc. B” according to | — | 0.25 | — | — |
| example 4 | | | | |
| Perfume oil according to | — | 1.00 | — | 0.50 |
| example 2 | | | | |
| “Acc. C” according to | — | — | 0.30 | 0.25 |
| example 4 | | | | |
| Perfume oil according to | — | — | 0.95 | — |
| example 3 | | | | |
| Water | To 100 | To 100 | To 100 | To 100 |

### Example 8

### Transparent Deo-Sticks (Formulations A, B) or Deo-Crème Sticks (Formulations C, D)

| | A | B | C | D |
|---|---|---|---|---|
| | % by | % by | % by | % by |
| Components | weight | weight | weight | weight |
| Aluminum zirconium | 25.00 | 20.00 | 25.00 | 20.00 |
| tetrahydrochlorate - glycin | | | | |
| Komplex | | | | |
| Dimethicon (10 cst) | — | — | 5.00 | 5.00 |
| Cyclopentasiloxane | — | 0.50 | 1.00 | 0.50 |
| Petrolatum | 5.00 | 4.70 | 5.00 | 5.00 |
| Ozokerite | 1.00 | 1.50 | — | — |
| Stearyl alcohol | 12.00 | 12.00 | — | — |
| 2-butyloctanic acid | 0.50 | — | 0.50 | — |
| Wax | — | — | 1.25 | 1.25 |
| PPG-14 butyl ether | 9.00 | 9.00 | — | — |
| Hardened rapeseed oil | — | — | 5.00 | 5.00 |
| Silicon dioxide | — | — | 1.00 | — |
| Farnesol | 0.25 | — | 0.25 | — |
| Paraffin oil | 0.50 | 0.50 | — | — |
| Hydrated castor oil (castor | 3.50 | 3.50 | — | — |
| wax) | | | | |
| Talc | 4.00 | 4.00 | — | — |
| Behenyl alcohol | 0.20 | 0.20 | — | — |
| d-panthenyl triacetate | 1.00 | 1.00 | — | — |
| Preservative (BHT) | 0.10 | 0.10 | 0.12 | — |
| Phenoxy ethanol | — | 0.15 | — | 0.50 |
| “Acc. A” according to | 0.20 | — | — | — |
| example 4 | | | | |
| Perfume oil according to | 0.90 | — | — | 0.50 |
| example 1 | | | | |
| “Acc. B” according to | — | 0.25 | — | — |
| example 4 | | | | |
| Perfume oil according to | — | 1.00 | — | 0.50 |
| example 2 | | | | |
| “Acc. C” according to | — | — | 0.30 | 0.25 |
| example 4 | | | | |
| Perfume oil according to | — | — | 0.95 | — |
| example 3 | | | | |
| Water | To 100 | To 100 | To 100 | To 100 |

### Example 9

### Roll-on Antiperspirant

| | | % by |
|---|---|---|
| Components | % by weight | weight |
| Caprylyl Trimethicon (SilCare TM Silicone | 0.30 | 0.30 |
| 31 M 50) | | |
| Steareth-20 (GENAPOL TM HS 200) | 3.00 | 3.00 |
| Steareth-2 (GENAPOL TM HS 020) | 1.50 | 1.50 |
| Dicaprylyl ether (Cetiol TM OE) | 2.00 | 2.00 |
| Coco caprylate/caprate (Cetiol TM LC) | 2.00 | 2.00 |
| Glycerine | 2.00 | 2.00 |
| Glyceryl stearate (Cutina TM GMS) | 2.00 | 2.00 |
| Octyldodecanol (Eutanol TM G) | 1.00 | 1.00 |
| Stearyl alcohol | 2.50 | 2.50 |
| Aluminum hydrochloride according to example | 10.00 | 10.00 |
| 1 of EP 1321431 | | |
| Avocado extract Persea gratissima | 0.30 | 0.20 |
| Chamomile extract | 0.10 | — |
| “Acc. B” according to example 4 | 0.20 | — |
| Perfume oil according to example 1 | 0.85 | — |
| “Acc. C” according to example 4 | — | 0.30 |
| Perfume oil according to example 3 | — | 1.00 |
| Water | To 100 | To 100 |

### Example 10

### Antiperspirant Stick

| | % by | % by | % by |
|---|---|---|---|
| Components | weight | weight | weight |
| Phenyl Trimethicon (SilCare TM Silicone | 13.50 | 13.50 | 13.50 |
| 15 M 50) | | | |
| Cetearyl alcohol | To 100 | To 100 | To 100 |
| Cetiol CC (dicaprylyl carbonate) | 13.50 | 13.50 | 13.50 |
| Stearic acid | 3.50 | 3.50 | 3.50 |
| PEG-40-hydrated castor oil (Emulsogen TM | 4.10 | 4.10 | 4.10 |
| HCO 040) | | | |
| PEG-8 distearate (Cithrol 4 DS) | 4.10 | 4.10 | 4.10 |
| Petrolatum | 6.90 | 6.90 | 6.90 |
| Aluminum hydrochlorate | 13.80 | 13.80 | 13.80 |
| Aluminum zirconium trichlorohydrex gly | 20.00 | 19.50 | 20.00 |
| Neo Helopan Hydro (phenylbenzimidazole | 2.00 | — | 3.50 |
| sulphonic acid, Symrise) | | | |
| 2.2-dimethyl-3-phenylpropanol (muguet | — | 0.25 | — |
| alcohol) | | | |
| Ethylhexylglycerine (octoxyglycerin) | — | 0.30 | 0.20 |
| 1,1-dimethyl-3-phenylpropanol | — | 0.25 | 0.10 |
| “Acc. B” according to example 4 | 0.20 | — | 0.25 |
| Perfume oil according to example 2 | 0.85 | — | — |
| “Acc. C” according to example 4 | — | 0.30 | — |
| Perfume oil according to example 3 | — | 1.00 | 0.95 |

### Example 11

### Antiperspirant Spray

| | % by | % by | % by |
|---|---|---|---|
| Components | weight | weight | weight |
| Glyceryl stearate | 2.50 | 2.50 | 2.50 |
| Cetearyl alcohol | 1.00 | 1.00 | 1.00 |
| Ceteareth-20 | 2.50 | 2.50 | 2.50 |
| Ceteareth-12 | 1.00 | 1.00 | 1.00 |
| Dicaprylyl ether | 5.00 | 5.00 | 5.00 |
| Coco caprylate/caprate | 5.00 | 5.00 | 5.00 |
| Caffeine | 0.25 | 0.25 | 0.25 |
| Phenoxy ethanol | 0.25 | 0.25 | 0.25 |
| Parabens (mixture of methyl-, ethyl-, | 0.25 | 0.25 | 0.25 |
| propyl-, butyl-, isobutylparaben) | | | |
| 1,2-pentandiol (Hydrolite-5) | 2.00 | 1.00 | 1.20 |
| Aluminum hydrochlorate | 8.00 | 8.00 | 8.00 |
| Neo Heliopan AP (disodium phenyl | 1.00 | — | 1.00 |
| dibenzimidazole tetrasulfonate, Symrise) | | | |
| 4-methyl-4-phenyl-2-pentanol (Vetikol) | — | 0.10 | 0.10 |
| Ethylhexylglycerine (octoxyglycerin) | — | 0.30 | 0.50 |
| “Acc. A” according to example 4 | 0.25 | — | — |
| Perfume oil according to example 1 | 0.90 | — | — |
| “Acc. B” according to example 4 | — | 0.30 | — |
| Perfume oil according to example 2 | — | 1.00 | — |
| “Acc. C” according to example 4 | — | — | 0.30 |
| Perfume oil according to example 3 | — | — | 0.95 |
| Water | To 100 | To 100 | To 100 |

### Example 12

### Aerosol Spray

| | % by | % by | % by |
|---|---|---|---|
| Components | weight | weight | weight |
| Octyldodecanol | 0.50 | — | 0.50 |
| Phenoxy ethanol | — | — | 0.30 |
| 1,2-pentandiol (Hydrolite-5) | 1.00 | 1.00 | 0.50 |
| 1,2-hexandiol | 0.25 | 0.15 | — |
| 1,2-octandiol | 0.25 | 0.25 | — |
| Farnesol | 0.25 | — | 0.25 |
| L-menthyllactate (Frescolat ML, Symrise) | 0.25 | 0.40 | 0.60 |
| 1,1-dimethyl-3-phenylpropanol | 0.30 | — | 0.10 |
| Ethylhexylglycerine (octoxyglycerin) | — | 0.30 | 0.50 |
| “Acc. A” according to example 4 | 0.20 | — | — |
| Perfume oil according to example 2 | 0.90 | — | — |
| “Acc. B” according to example 4 | — | 0.25 | — |
| Perfume oil according to example 1 | — | 1.00 | — |
| “Acc. C” according to example 4 | — | — | 0.30 |
| Perfume oil according to example 3 | — | — | 0.95 |
| Ethanol | To 100 | To 100 | To 100 |

The liquid mixture obtained following the mixing together of the stated components was filled with a propane-butane mixture (2:7 = in the ratio 2:3) in an aerosol container

### Example 13

### Pump Spray

| | % by | % by | % by |
|---|---|---|---|
| Components | weight | weight | weight |
| PEG-40 hydrated castor oil | 2.00 | 2.00 | 2.00 |
| Glycerine | 1.00 | 1.00 | 1.00 |
| 1,2-pentandiol (Hydrolite-5) | — | 1.00 | 2.00 |
| 1,2-hexandiol | 0.25 | 0.15 | — |
| 1,2-octandiol | 0.25 | 0.15 | — |
| Anis alcohol | 0.15 | 0.10 | 0.25 |
| 2-methyl-4-phenyl-2-butanol | 0.40 | 0.50 | 0.50 |
| Ethylhexylglycerine (octoxyglycerine) | — | — | 0.40 |
| “Acc. A” according to example 4 | 0.30 | — | — |
| Perfume oil according to example 2 | 0.85 | — | — |
| “Acc. B” according to example 4 | — | 0.25 | — |
| Perfume oil according to example 3 | — | 0.90 | — |
| “Acc. C” according to example 4 | — | — | 0.20 |
| Perfume oil according to example 1 | — | — | 1.05 |
| Water | To 100 | To 100 | To 100 |

### Example 14

### Roll-on Gel

| | % by | % by | % by |
|---|---|---|---|
| Components | weight | weight | weight |
| PEG-40 hydrated castor oil | 2.00 | 2.00 | 2.00 |
| 1,3-butylene glycol | 2.00 | 2.00 | 2.00 |
| Hydroxyethylcellulose | 0.50 | 0.50 | 0.50 |
| 1,2-pentandiol (Hydrolite-5) | — | 1.00 | 2.00 |
| 1,2-hexandiol | 0.25 | 0.15 | — |
| 1,2-octandiol | 0.25 | — | 0.25 |
| L-menthyllactate (Frescolat ML, Symrise) | 0.25 | — | 0.50 |
| Phenoxy ethanol | 0.15 | 0.10 | 0.25 |
| 1,1-dimethyl-3-phenylpropanol | 0.20 | 0.15 | 0.10 |
| Ethylhexylglycerine (octoxyglycerin) | 0.50 | — | |
| “Acc. A” according to example 4 | 0.20 | — | — |
| Perfume oil according to example 1 | 0.90 | — | — |
| “Acc. B” according to example 4 | — | 0.15 | — |
| Perfume oil according to example 3 | — | 1.10 | — |
| “Acc. C” according to example 4 | — | — | 0.35 |
| Perfume oil according to example 2 | — | — | 0.75 |
| Water | To 100 | To 100 | To 100 |

### Example 15

### Roll-on Emulsion

| | % by | % by | % by |
|---|---|---|---|
| Components | weight | weight | weight |
| Triceteareth-4 phosphate | 0.30 | 0.30 | 0.30 |
| Octyldodecanol | 2.00 | 2.00 | 2.00 |
| C₁₂-₁₅-alkylbenzoate | 2.00 | 2.00 | 2.00 |
| C₁₀-₃₀-alkyl acrylate | 0.15 | 0.15 | 0.15 |
| 1,2-hexandiol/1,2-octandiol (1:1) | — | 0.50 | 0.25 |
| (Symdiol 68) | | | |
| Phenoxy ethanol | 0.25 | 0.25 | — |
| Parabens | 0.25 | — | 0.20 |
| 4-methyl-4-phenyl-2-pentanol (Vetikol) | 0.10 | — | 0.10 |
| 1,1-dimethyl-3-phenylpropanol | 0.10 | 0.25 | — |
| Ethylhexylglycerine (octoxyglycerin) | 0.50 | — | — |
| “Acc. A” according to example 4 | 0.25 | — | — |
| Perfume oil according to example 1 | 0.90 | — | — |
| “Acc. B” according to example 4 | — | 0.30 | — |
| Perfume oil according to example 2 | — | 0.80 | — |
| “Acc. C” according to example 4 | — | — | 0.30 |
| Perfume oil according to example 3 | — | — | 0.85 |
| Water | To 100 | To 100 | To 100 |

### Example 16

### Wax Stick

| | % by | % by | % by |
|---|---|---|---|
| Components | weight | weight | weight |
| Hydrated castor oil | 5.00 | 5.00 | 5.00 |
| Beeswax | 6.00 | 6.00 | 6.00 |
| C₁₂-₁₅-alkylbenzoate | 17.00 | 17.00 | 17.00 |
| Ceresin | 30.00 | 30.00 | 30.00 |
| Neo Heliopan AV (ethylhexyl | 1.00 | 2.00 | — |
| methoxycinnamate, Symrise) | | | |
| Neo Heliopan 357 (butyl | 1.00 | — | 2.50 |
| methoxydibenzoylmethane, Symrise) | | | |
| 1,2-hexandiol/1,2-octandiol (1:1) | — | 0.50 | 0.25 |
| (Symdiol 68) | | | |
| Phenoxy ethanol | 0.50 | — | — |
| Parabens | — | — | 0.25 |
| 1,1-dimethyl-3-phenylpropanol | 0.20 | — | 0.10 |
| 2-methyl-4-phenyl-2-butanol | — | 0.50 | — |
| 2.2-dimethyl-3-phenylpropanol | 0.30 | — | 0.35 |
| (muguet alcohol) | | | |
| “Acc. B” according to example 4, | 1.00 | — | 0.50 |
| encapsulated in beta-cyclodextrin, | | | |
| content: 25% by weight | | | |
| Perfume oil according to example 1 | 0.80 | — | — |
| “Acc. A” according to example 4 | — | 0.25 | — |
| Perfume oil according to example 2 | — | 1.00 | — |
| “Acc. C” according to example 4 | — | — | 0.20 |
| Perfume oil according to example 3 | — | — | 0.95 |
| Octyldodecanol | To 100 | To 100 | To 100 |

### Example 17

### Deo Atomizer

| | % by | % by | % by |
|---|---|---|---|
| Components | weight | weight | weight |
| PEG-40 hydrated castor oil | 3.00 | 3.00 | 3.00 |
| Ethylhexylglycerine (octoxyglycerin) | 0.80 | 0.80 | 1.00 |
| Ethanol | 40.00 | 40.00 | 40.00 |
| Citrate buffer | 0.50 | 0.50 | 0.50 |
| 1,2-hexandiol/1,2-octandiol (1:1) | — | 0.25 | 0.25 |
| Phenoxy ethanol | 0.25 | 0.25 | — |
| Triclosan ® (5-chloro-2-(2,4- | 0.25 | — | 0.20 |
| dichlorphenoxy)phenol) | | | |
| 2-benzylheptan-1-ol (Jasmol) | — | 0.15 | 0.15 |
| 1,1-dimethyl-3-phenylpropanol | 0.30 | 0.40 | 0.50 |
| “Acc. A” according to example 4 | 0.25 | — | — |
| Perfume oil according to example 1 | 0.90 | — | — |
| “Acc. B” according to example 4 | — | 0.30 | — |
| Perfume oil according to example 2 | — | 0.85 | — |
| “Acc. C” according to example 4 | — | — | 0.20 |
| Perfume oil according to example 3 | — | — | 1.00 |
| Water | To 100 | To 100 | To 100 |

### Example 18

### Roll-on

| | % by | % by | % by |
|---|---|---|---|
| Components | weight | weight | weight |
| Hydroxyethylcellulose | 0.50 | 0.50 | 0.50 |
| Ethylhexylglycerine (octoxyglycerin) | 1.00 | 0.80 | 1.00 |
| Ethanol | 45.00 | 45.00 | 40.00 |
| Aluminum hydrochlorate | 15.00 | 15.00 | 20.00 |
| 1,2-hexandiol/1,2-octandiol (1:1) | — | 0.25 | 0.25 |
| Phenoxy ethanol | 0.25 | 0.25 | — |
| Parabens | 0.25 | — | 0.20 |
| 3-methyl-6-phenyl-2-hexanol | 0.40 | 0.15 | 0.20 |
| “Acc. A” according to example 4 | 0.20 | — | — |
| Perfume oil according to example 1 | 1.00 | — | — |
| “Acc. B” according to example 4 | — | 0.30 | — |
| Perfume oil according to example 3 | — | 0.80 | — |
| “Acc. C” according to example 4 | — | — | 0.35 |
| Perfume oil according to example 2 | — | — | 0.75 |
| Water | To 100 | To 100 | To 100 |

### Example 19

### Emulsion Roll-on

| | % by | % by | % by |
|---|---|---|---|
| Components | weight | weight | weight |
| PEG-40 stearate | 5.00 | 5.00 | 5.00 |
| Ethylhexylglycerine (octoxyglycerin) | 1.00 | 0.80 | 1.00 |
| Cetyl alcohol | 2.00 | 2.00 | 2.00 |
| Mineral oil | 2.00 | 2.00 | 2.00 |
| Aluminum hydrochlorate | 15.00 | 15.00 | 15.00 |
| Polysorbate 80 | 1.00 | 1.00 | 1.00 |
| Glycerine | 1.50 | 1.50 | 1.50 |
| Mg-aluminum silicate | 0.80 | 0.80 | 0.80 |
| 1,2-pentandiol (Hydrolite-5) | — | 0.50 | 1.00 |
| 2-benzylheptan-1-ol (Jasmol) | — | 0.25 | — |
| 2-methyl-4-phenylbutan-2-ol | 0.25 | — | 0.40 |
| “Acc. A” according to example 4 | 0.25 | — | — |
| Perfume oil according to example 2 | 0.90 | — | — |
| “Acc. B” according to example 4 | — | 0.30 | — |
| Perfume oil according to example 2 | — | 0.80 | — |
| “Acc. C” according to example 4 | — | — | 0.30 |
| Perfume oil according to example 2 | — | — | 0.85 |
| Water | To 100 | To 100 | To 100 |

### Example 20

### Roll-on

| | % by | % by | % by |
|---|---|---|---|
| Components | weight | weight | weight |
| Steareth-2 | 2.00 | 2.00 | 2.00 |
| Steareth-21 | 5.00 | 5.00 | 5.00 |
| Ethylhexylglycerine (octoxyglycerin) | 1.00 | 0.80 | 1.00 |
| Cetearyl alcohol | 0.50 | 0.50 | 0.50 |
| 1,2-pentandiol (Hydrolite-5) | — | 0.50 | 1.00 |
| L-menthylmethylether | — | 0.10 | 0.15 |
| 1,1-dimethyl-3-phenylpropanol | 0.40 | 0.30 | — |
| “Acc. A” according to example 4 | 0.25 | — | — |
| Perfume oil according to example 2 | 0.90 | — | — |
| “Acc. B” according to example 4 | — | 0.40 | — |
| Perfume oil according to example 2 | — | 0.80 | — |
| “Acc. C” according to example 4 | — | — | 0.30 |
| Perfume oil according to example 2 | — | — | 0.85 |
| Water | To 100 | To 100 | To 100 |

### Example 21

### Antiperspirant Aerosol

| | % by | % by | % by |
|---|---|---|---|
| Components | weight | weight | weight |
| Silicone | 13.00 | 13.00 | 13.00 |
| Ethylhexylglycerine (octoxyglycerin) | 0.80 | 0.80 | 1.00 |
| Ethanol | 0.80 | 0.80 | 0.80 |
| Quaternium-18 hectorite | 0.80 | 0.80 | 0.80 |
| Aluminum hydrochlorate, powder | 10.00 | 10.00 | 10.00 |
| 1,2-hexandiol/1,2-octandiol (1:1) | 0.50 | 0.25 | 0.40 |
| (Symdiol 68) | | | |
| “Acc. A” according to example 4 | 0.30 | — | — |
| Perfume oil according to example 3 | 1.10 | — | — |
| “Acc. B” according to example 4 | — | 0.30 | — |
| Perfume oil according to example 1 | — | 0.90 | — |
| “Acc. C” according to example 4 | — | — | 0.40 |
| Perfume oil according to example 1 | — | — | 0.70 |
| Water | To 100 | To 100 | To 100 |

Quaternium-18 hectorite (INCI-designation; CAS#: 71011-27-3):

quaternary ammonium compounds, bis(hydrated tallow-alkyl)dimethyl-, chloride, compounds with hectorite

### Example 22

### Suspensions Roll-on

| | % by | % by | % by |
|---|---|---|---|
| Components | weight | weight | weight |
| Silicone | To 100 | To 100 | To 100 |
| Ethylhexylglycerine (octoxyglycerin) | 1.00 | 1.00 | 1.00 |
| Quaternium-18 hectorite | 13.00 | 13.20 | 13.70 |
| Aluminum hydrochlorate, powder | 21.00 | 20.00 | 20.00 |
| 1,1-dimethyl-3-phenylpropanol | 0.25 | 0.50 | 0.50 |
| 4-methyl-4-phenyl-2-pentanol (Vetikol) | 0.10 | — | 0.10 |
| “Acc. A” according to example 4 | 0.25 | — | — |
| Perfume oil according to example 2 | 1.00 | — | — |
| “Acc. B” according to example 4 | — | 0.25 | — |
| Perfume oil according to example 3 | — | 0.85 | — |
| “Acc. C” according to example 4 | — | — | 0.40 |
| Perfume oil according to example 3 | — | — | 0.85 |

### Example 23

### Suspension Stick

| | % by | % by | % by |
|---|---|---|---|
| Components | weight | weight | weight |
| Stearyl alcohol | 20.00 | 20.00 | 20.00 |
| Cyclomethicone | To 100 | To 100 | To 100 |
| PPG-14 butyl ether | 2.00 | 2.00 | 2.00 |
| Hydrated castor oil | 1.00 | 1.00 | 1.00 |
| Talc | 2.00 | 2.00 | 2.00 |
| Aluminum hydrochlorate, powder | 20.00 | 20.00 | 20.00 |
| Triclosan ® (5-chloro-2-(2,4- | 0.30 | — | 0.30 |
| dichlorphenoxy)phenol) | | | |
| Ethylhexylglycerine (octoxyglycerin) | 0.50 | 0.80 | 0.50 |
| 1,1-dimethyl-3-phenylpropanol | 0.30 | 0.40 | 0.55 |
| 2.2-dimethyl-3-phenylpropanol (muguet | 0.30 | 0.15 | — |
| alcohol) | | | |
| Anis alcohol | — | — | 0.15 |
| “Acc. A” according to example 4 | 0.25 | — | — |
| Perfume oil according to example 1 | 0.90 | — | — |
| “Acc. B” according to example 4 | — | 0.35 | — |
| Perfume oil according to example 2 | — | 0.75 | — |
| “Acc. C” according to example 4 | — | — | 0.50 |
| Perfume oil according to example 3 | — | — | 0.65 |

### Example 24

### Antiperspirant Formulations

| | % by | % by | % by |
|---|---|---|---|
| Components | weight | weight | weight |
| Reach AZP-908 SUF | 24.00 | 22.00 | 20.00 |
| Cyclomethicone (Pentamer) | To 100 | To 100 | To 100 |
| Polydecene (Silkflo 364 NF) | 17.50 | 20.00 | 20.00 |
| Neo Helipan OS (ethylhexyl salicylate, | 2.50 | 1.00 | — |
| Symrise) | | | |
| L-menthyllactate (Frescolat ML, Symrise) | 0.25 | — | 0.50 |
| Polyethylene | 3.00 | 3.00 | 3.00 |
| Hydrated castor oil | 2.00 | 2.00 | 2.00 |
| Promyristyl PM-3 | 7.00 | 7.00 | 7.00 |
| PEG-8 distearate | 3.00 | 3.00 | 3.00 |
| Silicon dioxide (Cab-O-Sil M-5) | 1.00 | 1.00 | 1.00 |
| Stearyl alcohol | 15.00 | 10.00 | 15.00 |
| Octyldodecanol | — | 8.00 | — |
| “Acc. A” according to example 4 | 0.25 | — | — |
| Perfume oil according to example 1 | 0.90 | — | — |
| “Acc. B” according to example 4 | — | 0.30 | — |
| Perfume oil according to example 2 | — | 0.80 | — |
| “Acc. C” according to example 4 | — | — | 0.30 |
| Perfume oil according to example 3 | — | — | 0.85 |

Reach AZP-908 SUF: activated aluminum zirconium tetrachlorohydrex glycine - complex, a product of Reheis Inc.

### Example 25

### Impregnation Medium for Deo-Cloths

| | % by | % by | % by |
|---|---|---|---|
| Components | weight | weight | weight |
| PEG-40 hydrated castor oil | 3.00 | 3.00 | 3.00 |
| Octoxyglycerine | 1.00 | 0.80 | 0.90 |
| 2-methyl-4-phenylbutan-2-ol | 0.80 | 0.60 | 0.40 |
| Citrate buffer | 0.50 | 0.50 | 0.50 |
| Ethanol | 40.00 | 40.00 | 40.00 |
| “Acc. A” according to example 4 | 0.15 | — | — |
| Perfume oil according to example 1 | 0.50 | — | — |
| “Acc. B” according to example 4 | — | 0.20 | — |
| Perfume oil according to example 2 | — | 0.80 | — |
| “Acc. C” according to example 4 | — | — | 0.15 |
| Perfume oil according to example 1 | — | — | 0.70 |
| Water | To 100 | To 100 | To 100 |

### Example 26

### PIT Emulsion

| | % by | % by | % by |
|---|---|---|---|
| Components | weight | weight | weight |
| Glyceryl stearate, ceteareth-20, ceteareth-10, | 4.50 | 4.50 | 4.50 |
| cetearyl alcohol, cetyl palmitate | | | |
| Octoxyglycerine | 1.00 | 0.80 | 0.90 |
| Ceteareth-20 | 1.00 | 1.00 | 1.00 |
| Dicaprylyl ether | 5.00 | 5.00 | 5.00 |
| Dioctylcyclohexane | 5.00 | 5.00 | 5.00 |
| Aluminum hydrochlorate | 5.00 | 5.00 | 5.00 |
| “Acc. A” according to example 4 | 0.20 | — | — |
| Perfume oil according to example 1 | 0.50 | — | — |
| “Acc. B” according to example 4 | — | 0.20 | — |
| Perfume oil according to example 2 | — | 0.60 | — |
| “Acc. C” according to example 4 | — | — | 0.15 |
| Perfume oil according to example 1 | — | — | 0.70 |
| Water | To 100 | To 100 | To 100 |

### Example 27

### Micro-Emulsion

| | % by | % by | % by |
|---|---|---|---|
| Components | weight | weight | weight |
| Glycerine isostearate | 1.80 | 2.00 | 1.80 |
| Octoxyglycerine | 1.00 | 0.80 | 0.90 |
| Ceteareth-15 | 5.20 | 5.50 | 5.00 |
| Isotridecylisononanoate | 3.30 | 3.50 | 3.80 |
| Cyclomethicon | 6.60 | 6.40 | 6.20 |
| “Acc. A” according to example 4 | 0.20 | — | — |
| Perfume oil according to example 1 | 0.50 | — | — |
| “Acc. B” according to example 4 | — | 0.20 | — |
| Perfume oil according to example 2 | — | 0.60 | — |
| “Acc. C” according to example 4 | — | — | 0.15 |
| Perfume oil according to example 3 | — | — | 0.60 |
| Water | To 100 | To 100 | To 100 |

### Example 28

### Micro-Emulsion Gel

| | % by | % by | % by |
|---|---|---|---|
| Components | weight | weight | weight |
| Glycerine isostearate | 1.80 | 2.00 | 1.80 |
| Octoxyglycerine | 1.00 | 0.80 | 0.90 |
| Ceteareth-15 | 5.20 | 5.00 | 5.50 |
| PEG-150 distearate | 1.00 | 1.00 | 1.00 |
| Aluminum hydrochlorate | 5.00 | 5.00 | 5.00 |
| Isotridecylisononanoate | 3.30 | 3.50 | 3.80 |
| Cyclomethicon | 6.60 | 6.40 | 6.20 |
| “Acc. A” according to example 4 | 0.20 | — | — |
| Perfume oil according to example 2 | 0.50 | — | — |
| “Acc. B” according to example 4 | — | 0.20 | — |
| Perfume oil according to example 3 | — | 0.60 | — |
| “Acc. C” according to example 4 | — | — | 0.15 |
| Perfume oil according to example 2 | — | — | 0.70 |
| Water | To 100 | To 100 | To 100 |

### Example 29

### Deo-Stick

| | A | B | C |
|---|---|---|---|
| | % by | % by | % by |
| Components | weight | weight | weight |
| Sodium stearate | 8.00 | 8.00 | 8.00 |
| PPG-3 myristyl ether | 70.00 | 70.00 | 70.00 |
| 1,2-propylene glycol | 10.00 | 10.00 | 10.00 |
| 1,1-dimethyl-3-phenylpropanol | 0.50 | 0.25 | 0.30 |
| 2-butyloctanic acid | — | 0.20 | 0.10 |
| “Acc. A” according to example 4 | 0.10 | — | — |
| Perfume oil according to example 1 | 0.45 | — | — |
| “Acc. B” according to example 4 | — | 0.15 | — |
| Perfume oil according to example 2 | — | 0.50 | — |
| “Acc. C” according to example 4 | — | — | 0.25 |
| Perfume oil according to example 3 | — | — | 0.45 |
| Water | To 100 | To 100 | To 100 |

### Example 30

### Antiperspirant Stick

| | % by | % by | % by |
|---|---|---|---|
| Components | weight | weight | weight |
| Octyldodecanol | 8.00 | 7.00 | 8.50 |
| C20-40 pareth 10 | 0.50 | 0.50 | 0.50 |
| C20-40 pareth 40 | 1.50 | 1.50 | 1.50 |
| Stearyl alcohol | 4.00 | 4.00 | 3.00 |
| Hydrated castor oil | 2.00 | 2.00 | 2.00 |
| Octylisononanoate | 9.00 | 9.50 | 8.50 |
| Polydecene (Silkflo 364 NF) | 9.00 | 9.50 | 8.50 |
| PEG-14 butyl ether | 4.50 | 4.80 | 5.00 |
| Paraffin oil | 5.00 | 4.80 | 4.50 |
| Reach AZP-908 SUF (see example 24) | 24.00 | 24.00 | 24.00 |
| Tween-80 | 0.80 | 0.80 | 0.80 |
| Cyclomethicon | To 100 | To 100 | To 100 |
| Bentone gel VS-5 PC | 0.50 | 0.50 | 0.50 |
| Bishydrated tallow glyceride | 8.00 | 5.00 | — |
| “Acc. A” according to example 4 | 0.30 | — | — |
| Perfume oil according to example 1 | 0.80 | — | — |
| “Acc. B” according to example 4 | — | 0.20 | — |
| Perfume oil according to example 2 | — | 0.80 | — |
| “Acc. C” according to example 4 | — | — | 0.25 |
| Perfume oil according to example 3 | — | — | 0.65 |
| Water | To 100 | To 100 | To 100 |

### Example 31

### Hair Crème

| | % by | % by | % by |
|---|---|---|---|
| Components | weight | weight | weight |
| Vaseline | 4.00 | 4.00 | 4.00 |
| Cetearyl alcohol | 4.00 | 4.00 | 4.00 |
| PEG-40 hydrated castor oil | 2.00 | 2.00 | 2.00 |
| Isopropylpalmitate | 5.00 | 5.00 | 5.00 |
| Citric acid | 5.00 | 5.00 | 5.00 |
| Phenoxy ethanol | 0.30 | 0.60 | 0.70 |
| BHT | 0.10 | — | — |
| “Acc. A” according to example 4 | 0.20 | — | — |
| Perfume oil according to example 1 | 0.50 | — | — |
| “Acc. B” according to example 4 | — | 0.20 | — |
| Perfume oil according to example 2 | — | 0.60 | — |
| “Acc. C” according to example 4 | — | — | 0.15 |
| Perfume oil according to example 3 | — | — | 0.55 |
| Water | To 100 | To 100 | To 100 |

### Example 32

### Foot Crème

| | % by | % by | % by |
|---|---|---|---|
| Components | weight | weight | weight |
| Soluan 5 | 2.00 | 2.00 | 2.00 |
| Methylsalicylate | 1.00 | 1.00 | 1.00 |
| Coco caprylate/caprate | 10.00 | 10.00 | 10.00 |
| Stearic acid | 5.00 | 5.00 | 5.00 |
| Cetyl alcohol | 1.00 | 1.00 | 1.00 |
| Glycerine | 2.00 | 2.00 | 2.00 |
| Dimethicon | 1.00 | 1.00 | 1.00 |
| Carbopol 984 | 0.50 | 0.50 | 0.50 |
| Triethanolamine | 1.50 | 1.50 | 1.50 |
| Phenoxy ethanol | 0.50 | — | 0.30 |
| 1,2-pentandiol (Hydrolite-5, Symrise) | 0.50 | 2.00 | 1.50 |
| “Acc. A” according to example 4 | 0.30 | — | — |
| Perfume oil according to example 1 | 0.70 | — | — |
| “Acc. B” according to example 4 | — | 0.40 | — |
| Perfume oil according to example 2 | — | 0.80 | — |
| “Acc. C” according to example 4 | — | — | 0.50 |
| Perfume oil according to example 3 | — | — | 0.70 |
| Water | To 100 | To 100 | To 100 |

### Example 33

### Syndet Soap

| | % by | % by | % by |
|---|---|---|---|
| Components | weight | weight | weight |
| Sodium lauryl sulphate | 30.00 | 30.00 | 30.00 |
| Sodium sulphosuccinate | 10.00 | 10.00 | 10.00 |
| Potassium cocoyl hydrolyzed collagen | 2.00 | 2.00 | 2.00 |
| Dimethicon copolyol | 2.00 | 2.00 | 2.00 |
| Paraffin | 2.00 | 2.00 | 2.00 |
| Maize starch | 10.00 | 10.00 | 10.00 |
| Talcum | 10.00 | 10.00 | 10.00 |
| Glycerine | 3.00 | 3.00 | 3.00 |
| Phenoxy ethanol | 0.50 | 0.50 | 0.50 |
| “Acc. A” according to example 4 | 0.30 | — | — |
| Perfume oil according to example 1 | 0.70 | — | — |
| “Acc. B” according to example 4 | — | 0.40 | — |
| Perfume oil according to example 1 | — | 0.80 | — |
| “Acc. C” according to example 4 | — | — | 0.50 |
| Perfume oil according to example 2 | — | — | 0.70 |
| Water | To 100 | To 100 | To 100 |

### Example 34

### O/W Lotion

| | % by | % by | % by |
|---|---|---|---|
| Components | weight | weight | weight |
| Paraffin oil | 5.00 | 5.00 | 5.00 |
| Isopropylpalmitate | 5.00 | 5.00 | 5.00 |
| Cetyl alcohol | 2.00 | 2.00 | 2.00 |
| Beeswax | 2.00 | 2.00 | 2.00 |
| Ceteareth-20 | 2.00 | 2.00 | 2.00 |
| PEG-20-glyceryl stearate | 1.50 | 1.50 | 1.50 |
| Glycerine | 3.00 | 3.00 | 3.00 |
| Phenoxy ethanol | 0.50 | 0.50 | — |
| Parabens (mixture of methyl-, ethyl-, | — | — | 0.30 |
| propyl-, butyl-, isobutylparaben) | | | |
| “Acc. A” according to example 4 | 0.20 | — | — |
| Perfume oil according to example 2 | 0.60 | — | — |
| “Acc. B” according to example 4 | — | 0.10 | — |
| Perfume oil according to example 3 | — | 0.50 | — |
| “Acc. C” according to example 4 | — | — | 0.30 |
| Perfume oil according to example 1 | — | — | 0.70 |
| Water | To 100 | To 100 | To 100 |

### Example 35

### Hair Conditioner with UV Protection

| | | % by | % by |
|---|---|---|---|
| Components | INCI Name | weight | weight |
| Lanette O | Cetearyl Alcohol | 4.00 | 4.00 |
| Dragoxat 89 | Ethylhexyl Isononanoate | 4.00 | 4.00 |
| Emulsiphos | Potassium Cetyl Phosphate, | 0.50 | 0.50 |
| | Hydrogenated Palm | | |
| | Glycerides | | |
| Natrosol 250 HR | Hydroxyethylcellulose | 0.25 | 0.25 |
| Neo Heliopan Hydro | Phenylbenzimidazole | 2.00 | 2.00 |
| | Sulphonic Acid | | |
| L-Arginin | Arginine | 1.20 | 1.20 |
| Benzophenon-4 | Benzophenone-4 | 0.50 | 0.50 |
| Neo Heliopan AP | Disodium Phenyl | 0.50 | 1.00 |
| | Dibenzimidazole | | |
| | Tetrasulfonate | | |
| Edeta BD | Disodium EDTA | 0.05 | 0.05 |
| Dragocide Liquid | Phenoxyethanol (and) | 0.80 | 0.80 |
| | Methylparaben (and) | | |
| | Butyparaben (and) | | |
| | Ethyparaben | | |
| | (and) Propylparaben | | |
| Dow Corning 949 | Amodimethicone, | 2.00 | 2.00 |
| Cationic Emulsion | Cetrimonium Chloride, | | |
| | Trideceth-12 | | |
| Dow Corning 5200 | Laurylmethicone Copolyol | 0.50 | 0.50 |
| “Acc. B” according to | Parfum | 0.25 | — |
| example 4 | | | |
| Perfume oil according to | Parfum | 0.95 | — |
| example 3 | | | |
| “Acc. C” according to | Parfum | — | 0.20 |
| example 4 | | | |
| Perfume oil according to | Parfum | | 0.80 |
| example 1 | | | |
| Water | Water (Aqua) | To 100 | To 100 |

### Example 36

### Hair Conditioner with UV Protection

| | | % by | % by |
|---|---|---|---|
| Components | INCI Name | weight | weight |
| Renex PEG 6000 | PEG-150 | 2.50 | 2.50 |
| Hair Conditioner Base | Cetyl Alcohol, | 3.00 | 3.00 |
| | Behentrimonium | | |
| | Chloride, Triticum Vulgare | | |
| | (Wheat) Bran Extract, | | |
| | Linoleic Acid | | |
| PCL-Solid | Stearyl Heptanoate, Stearyl | 0.50 | 0.50 |
| | Caprylate | | |
| Dow Corning 5200 | Laurylmethicone Copolyol | 0.50 | 0.50 |
| Natrosol 250 HR | Hydroxyethylcellulose | 0.50 | 0.50 |
| Benzophenon-4 | Benzophenone-4 | 1.00 | 0.50 |
| Neo Heliopan AP | Disodium Phenyl | 1.00 | 0.80 |
| | Dibenzimidazole | | |
| | Tetrasulfonate | | |
| Amino Methyl propanol | Amino Methyl propanol | 2.00 | 1.20 |
| Nipagin M | Methylparaben | 0.30 | 0.30 |
| Dow Corning 949 | Amodimethicone, | 2.00 | 2.00 |
| Cationic Emulsion | Cetrimonium Chloride, | | |
| | Trideceth-12 | | |
| “Acc. according to | Parfum | 0.25 | — |
| example 4 | | | |
| Perfume oil according to | Parfum | 0.90 | — |
| example 2 | | | |
| “Acc. C” according to | Parfum | — | 0.20 |
| example 4 | | | |
| Perfume oil according to | Parfum | | 0.80 |
| example 3 | | | |
| Water | Water (Aqua) | To 100 | To 100 |

All the preparations described in examples 7 to 34, which contained accords according to the invention, in each case demonstrated under identical observations to those described in examples 5 and/or 6, a good masking of the perception of the smell of sweat and gave the test subjects, and also the assessing members of the sensory panel, a sense of hygiene and cleanliness (of the test subjects). This impression was significantly stronger when each of the cosmetic preparations according to the invention was compared with cosmetic preparations with identical components except for the accord according to the invention.

### Specific Embodiments

Specific embodiment one comprises an accord, comprising, essentially consisting or consisting of:

i) ethylene brassylate,
(ii) alpha-isomethyl ionone,
iii) 2-methyl-5-phenylpentan-1-ol, and
iv) menthyl acetate,
as well as possibly one, two, three, four or all of components v) to ix):
v) alpha-n-methyl ionone,
vi) isomenthyl acetate,
vii) vanillin,
viii) neomenthyl acetate,
ix) neoisomenthyl acetate.

Specific embodiment two comprises the accord as in specific embodiment one, wherein the weight ratio of
i) ethylene brassylate to the total mass of components ii)-iv) is at least 40:100, more preferably 50:100;
   and/or
   iii) 2-methyl-5-phenylpentan-1-ol to the total mass of components i), ii) and iv) is at least 15:100, more preferably 20:100;
   and/or
iv) menthyl acetate to the total mass of components i)-iii) is at least 15:100, more preferably 20:100.

Specific embodiment three comprises the accord as in specific embodiment one or two, wherein

i) ethylene brassylate,
ii) alpha-isomethyl ionone,
v) alpha-n-methyl ionone,
iii) 2-methyl-5-phenylpentan-1-ol,
iv) menthyl acetate, and
vii) vanillin,
and one, two or all of components yl), vii) and/or ix):
vi) isomenthyl acetate,
viii) neomenthyl acetate,
ix) neoisomenthyl acetate
are included.

Specific embodiment four comprises a mixture, comprising an accord as in one of the above specific embodiments and at least one further fragrance.

Specific embodiment five comprises a perfume oil or cosmetic preparation, comprising an accord as in one of specific embodiments one to three or a mixture as in specific embodiment four.

Specific embodiment six comprises the cosmetic preparation as in specific embodiment five, comprising at least one fragrance from group (B), wherein group (B) consists of:

alpha-hexylcinnamaldehyde, 2-phenoxyethylisobutyrate (Phenirat), dihydromyrcenol (2,6-dimethyl-7-octen-2-ol), methyl dihydrojasmonate; preferably with a content of cis-isomers of >60 by weight (Hedione, Hedione HC), 4,6,6,7,8,8-hexamethyl-1,3,4,6,7,8-hexahydrocyclopenta[g]benzopyran (Galaxolid), tetrahydrolinalool (3,7-dimethyloctan-3-ol), ethyllinalool, benzylsalicylate, 2-methyl-3-(4-tert-butyl-phenyl)propanal (Lilial), cinnamon alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat), citronellol, linalyl acetate, styrolyl acetate(1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super), hexylsalicylate, 4-tert.-butylcyclohexyl acetate (Oryclon), 2-tert.-butylcyclohexyl acetate (Agrumex HC), alpha-ionone (4-(2,2,6-trimethyl-2-cyclohexen-1-yl)-3-buten-2-one), coumarin, terpinyl acetate, 4-(4-hydroxy-4-methylpentyl)-3-cyclohexene carboxaldehyde (Lyral), alpha-amyl cinnamon aldehyde, (E)- and/or (Z)-3-methylcyclopentadec-5-enone (Muscenon), 15-pentadec-11-enolide and/or 15-pentadec-12-enolide (Globalide), 15-cyclopentadecanolide (Macrolide), 1-(5,6,7,8-tetrahydro-3,5,5,6,8,8-hexamethyl-2-naphthalenyl)ethanone (Tonalid), 2-isobutyl-4-methyltetrahydro-2H-pyran-4-ol (Florol), 2-ethyl-4-(2,2,3-trimethyl-3-cyclopenten-1-yl)-2-buten-1-ol (Sandolen), menthol (preferably l-menthol or racemic menthol, with particular preference for l-menthol), anethole, geraniol, nerol, linalool, citronellol, linalyl acetate, 2-phenylethyl alcohol, 2,2-dimethyl-3-(3-methylphenyl)-propanol (Majantol), rose oxide (4-methyl-2-(2-methyl-1-propenyl)tetrahydropyran), allyl heptanoate, 4-methylacetophenone, (4aR,5R,7aS,9R)-octahydro-2,2,5,8,8,9a-hexamethyl-4H-4a,9-methanoazuleno(5,6-d)-1,3-dioxol) (Ambrocenide), Timberol (1-(2,2,6-trimethylcyclohexyl)hexan-3-ol), Floropal (2,4,6-trimethyl-4-phenyl-1,3-dioxan), benzylacetone, methyl cinnamate, 3a,6,6,9a-tetramethyldodecahydronaphtho[2,1-b]furan (Ambroxid).

Specific embodiment seven comprises the cosmetic preparation as in either of specific embodiments five or six, comprising at least one yielding agent selected from the group (H) consisting of:

(H): dipropylene glycol (DPG), diethylphthalate (DEP), triethylcitrate (TEC), isopropylmyristate (IPM), and/or benzylbenzoate (BB)
and/or a cooling agent.

Specific embodiment eight comprises the cosmetic preparation of one of specific embodiments five to seven wherein the cosmetic preparation is present in a form of application selected from the group comprising sticks, crèmes, gels, lotions, foams, roll-on preparations, powder sprays, aerosol or non-aerosol sprays.

Specific embodiment nine comprises the cosmetic preparation as in one of specific embodiments five to eight, wherein the preparation is a deodorant or an antiperspirant.

Specific embodiment ten comprises the use of an accord as in any of specific embodiments one to three, a mixture as in specific embodiment four, a perfume oil as in specific embodiment five, or a cosmetic preparation as in one of specific embodiments five to nine to generate a sense of hygiene and/or cleanliness.

Specific embodiment eleven comprises the use as in specific embodiment ten, wherein the sense of hygiene and/or cleanliness is generated through the interaction of the accord as in one of specific embodiments one to three

(i) with a body odor, wherein the inherent olfactory effect of the body odor is weakened and/or
(ii) with a fragrance, wherein the olfactory effect of the fragrance is strengthened.

Specific embodiment twelve comprises the use as in specific embodiment ten or eleven, wherein the body odor is the smell of human sweat, in particular the smell of underarm sweat.

Specific embodiment thirteen comprises a method for the cosmetic generation of a sense of hygiene and/or cleanliness, comprising the following steps:

a) Preparation (i) of an accord as in one of specific embodiments one to three or
(ii) of a mixture as in specific embodiment four or
(iii) of a perfume oil as in specific embodiment five or
(iv) of a preparation as in one of specific embodiments five to nine and
   b) Application of the accord, the mixture, the perfume oil or the preparation, so that the olfactory effect of the accord, the mixture, the perfume oil or the preparation is perceptible enough that a sense of hygiene and/or cleanliness is generated.

Specific embodiment fourteen comprises the method as in specific embodiment thirteen, wherein the application is to the skin.

Specific embodiment fifteen comprises the method as in specific embodiment thirteen or fourteen, wherein the application is by spraying.

1. An accord, comprising, essentially consisting of, or consisting of:
i) ethylene brassylate,
(ii) alpha-isomethyl ionone,
iii) 2-methyl-5-phenylpentan-1-ol, and
iv) menthyl acetate,
as well as possibly one, two, three, four or all of components v) to ix):
v) alpha-n-methyl ionone,
vi) isomenthyl acetate,
vii) vanillin,
viii) neomenthyl acetate,
ix) neoisomenthyl acetate.

2. The accord as claimed in claim 1, wherein the weight ratio of
i) ethylene brassylate to the total mass of components ii)-iv) is at least 40:100;
and/or
iii) 2-methyl-5-phenylpentan-1-ol to the total mass of components i), ii) and iv) is at least 15:100;
and/or
iv) menthyl acetate to the total mass of components i)-iii) is at least 15:100.

3. The accord as claimed in claim 1, wherein
i) ethylene brassylate,
ii) alpha-isomethyl ionone,
v) alpha-n-methyl ionone,
iii) 2-methyl-5-phenylpentan-1-ol,
iv) menthyl acetate, and
vii) vanillin,
and one, two or all of components yl), vii) and/or ix):
vi) isomenthyl acetate,
viii) neomenthyl acetate,
ix) neoisomenthyl acetate
are included.

4. A mixture, comprising an accord as claimed in claim 1 and at least one further fragrance.

5. A perfume oil or cosmetic preparation, comprising an accord as claimed in claim 1 or a mixture comprising an accord as claimed in claim 1 and at least one further fragrance.

6. A cosmetic preparation as claimed in claim 5, comprising at least one fragrance from group (B), wherein group (B) consists of:
alpha-hexylcinnamaldehyde, 2-phenoxyethylisobutyrate, dihydromyrcenol (2,6-dimethyl-7-octen-2-ol), methyl dihydrojasmonate, 4,6,6,7,8,8-hexamethyl-1,3,4,6,7,8-hexahydrocyclopenta[g]benzopyran, tetrahydrolinalool (3,7-dimethyloctan-3-ol), ethyllinalool, benzylsalicylate, 2-methyl-3-(4-tert-butyl-phenyl)propanal, cinnamon alcohol, 4,7-methano-3a,4,5,6,7,7a-hexahydro-5-indenyl acetate and/or 4,7-methano-3a,4,5,6,7,7a-hexahydro-6-indenyl acetate, citronellol, linalyl acetate, styrolyl acetate(1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene, hexylsalicylate, 4-tert.-butylcyclohexyl acetate, 2-tert.-butylcyclohexyl acetate, alpha-ionone (4-(2,2,6-trimethyl-2-cyclohexen-1-yl)-3-buten-2-one), coumarin, terpinyl acetate, 4-(4-hydroxy-4-methylpentyl)-3-cyclohexene carboxaldehyde, alpha-amyl cinnamon aldehyde, (E)- and/or (Z)-3-methylcyclopentadec-5-enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, menthol, anethole, geraniol, nerol, linalool, citronellol, linalyl acetate, 2-phenylethyl alcohol, 2,2-dimethyl-3-(3-methylphenyl)-propanol, rose oxide (4-methyl-2-(2-methyl-1-propenyl)tetrahydropyran), allyl heptanoate, 4-methylacetophenone, (4aR,5R,7aS,9R)-octahydro-2,2,5,8,8,9a-hexamethyl-4H-4a,9-methanoazuleno(5,6-d)-1,3-dioxol), 1-(2,2,6-trimethylcyclohexyl)hexan-3-ol, 2,4,6-trimethyl-4-phenyl-1,3-dioxan, benzylacetone, methyl cinnamate, and 3a,6,6,9a-tetramethyldodecahydronaphtho[2,1-b]furan.

7. The cosmetic preparation as claimed in claim 5, comprising at least one yielding agent selected from the group (H) consisting of:
(H): dipropylene glycol (DPG), diethylphthalate (DEP), triethylcitrate (TEC), isopropylmyristate (IPM), and benzylbenzoate (BB)
and/or a cooling agent.

8. The cosmetic preparation of claim 5 wherein the cosmetic preparation is present in a form of application selected from the group comprising sticks, crèmes, gels, lotions, foams, roll-on preparations, powder sprays, aerosol or non-aerosol sprays.

9. The cosmetic preparation as claimed in claim 5, wherein the preparation is a deodorant or an antiperspirant.

10. A method of generating a sense of hygiene and/or cleanliness comprising applying an accord as claimed in claim 1; a mixture comprising an accord as claimed in claim 1 and at least one further fragrance; a perfume oil comprising an accord as claimed in claim 1 or a mixture comprising an accord as claimed in claim 1 and at least one further fragrance; or a cosmetic preparation comprising an accord as claimed in claim 1 or a mixture comprising an accord as claimed in claim 1 and at least one further fragrance.

11. The method as claimed in claim 10, wherein the sense of hygiene and/or cleanliness is generated through the interaction of the accord
(i) with a body odor, wherein the inherent olfactory effect of the body odor is weakened and/or
(ii) with a fragrance, wherein the olfactory effect of the fragrance is strengthened.

12. The method as claimed in 10, wherein the body odor is the smell of human sweat.

13. A method for the cosmetic generation of a sense of hygiene and/or cleanliness, comprising the following steps:
a) Preparation (i) of an accord as claimed in claim 1 or
(ii) of a mixture comprising an accord as claimed in claim 1 and at least one further fragrance or
(iii) of a perfume oil comprising an accord as claimed in claim 1 or a mixture comprising an accord as claimed in claim 1 and at least one further fragrance or
(iv) of a preparation comprising an accord as claimed in claim 1 or a mixture comprising an accord as claimed in claim 1 and at least one further fragrance and
b) Application of the accord, the mixture, the perfume oil or the preparation, so that the olfactory effect of the accord, the mixture, the perfume oil or the preparation is perceptible enough that a sense of hygiene and/or cleanliness is generated.

14. The method as claimed in claim 13, wherein the application is to the skin.

15. The method as claimed in claim 13, wherein the application is by spraying.

16. The accord as claimed in claim 2 wherein the weight ratio of ethylene brassylate to the total mass of components ii)-iv) is at least 50:100.

17. The accord as claimed in claim 2 wherein the weight ratio of 2-methyl-5-phenylpentan-1-ol to the total mass of components i), ii) and iv) is at least 20:100.

18. The accord as claimed in claim 2 wherein the weight ratio of menthyl acetate to the total mass of components i)-iii) is at least 20:100.

19. The method as claimed in claim 12, wherein the body odor is the smell of underarm sweat.
